(19) United States
(12) Patent Application Publication
(10) Pub. No.: US 2025/0025516 A1
(43) Pub. Date: Jan. 23, 2025

(54) COMPOSITIONS AND METHODS FOR INHIBITING SEIZURES

(71) Applicant: The Regents of the University of California, Oakland, CA (US)

(72) Inventors: Elaine Y Hsiao, Los Angeles, CA (US); Gregory Lum, Los Angeles, CA (US)

(21) Appl. No.: 18/713,947

(22) PCT Filed: Dec. 2, 2022
(86) PCT No.: PCT/US22/51629

§ 371 Date: May 28, 2024

Related U.S. Application Data
(60) Provisional application No. 63/285,267, filed on Dec. 2, 2021.

(51) Int. Cl. A61K 35/744; A61K 35/742; A61K 45/06; A61P 25/08

(52) U.S. Cl. CPC A61K 35/744; A61K 35/742; A61K 45/06; A61P 25/08

(57) ABSTRACT

Provided herein are methods and compositions related to treating or preventing seizures.

### CROSS-REFERENCE TO RELATED APPLICATIONS

This application claims the benefit of priority to U.S. Provisional Application Ser. No. 63/285,267, filed on Dec. 2, 2021, the entire contents of which are incorporated herein in their entirety.

### GOVERNMENT SUPPORT

This invention was made with government support under Grant Number NS115537, awarded by the National Institutes of Health. The government has certain rights in the invention.

### BACKGROUND

Epilepsy is characterized by recurrent seizures that can lead to loss of awareness, loss of consciousness, and/or disturbances of movement, autonomic function, sensation (including vision, hearing and taste), mood, and/or mental function. Epilepsy afflicts 1-2% of the population in the developed world.

The low-carbohydrate, high-fat ketogenic diet (KD) is a treatment for refractory epilepsy, wherein more than one-third of epileptic individuals do not respond to existing anticonvulsant medications. The efficacy of the KD is supported by multiple retrospective and prospective studies, which estimate that ˜30% of patients become seizure-free, and ˜60% experience significant benefit. However, despite its value for treating epilepsy and its increasing application to other disorders, including autism, Alzheimer's disease, Parkinson's disease, metabolic syndrome and cancer, use of the KD remains low due to difficulties with implementation, dietary compliance and adverse side effects. In fact, even with successful seizure reduction, epileptic patient retention on the KD is only an estimated 12% by the third year of dietary therapy. Moreover, mechanisms underlying the beneficial effects of the KD are poorly understood, and molecular and/or cellular targets for intervention are lacking. That the diet succeeds in controlling various types of symptoms in cases when drugs fail suggests that it enhances endogenous neuroprotective pathways that are not targeted by existing medications.

### SUMMARY

Provided herein are methods and compositions for mimicking the effects of a ketogenic diet by administering compositions (e.g., the composition disclosed herein) to a subject. In certain embodiments, the methods and compositions are for the treatment or prevention of seizures in a subject (e.g., a subject with a neurodevelopmental disorder, such as autism spectrum disorder, Rett syndrome, fragile X, attention deficit disorder (ADD), attention deficit/hyperactivity disorder (ADHD), refractory epilepsy, such as pediatric refractory epilepsy, and/or non-refractory epilepsy). In other embodiments, the methods and compositions are for preventing or treating a condition (e.g., epilepsy, seizures, autism spectrum disorder, Alzheimer's disease, Huntington's disease, Parkinson's disease, amyotrophic lateral sclerosis (ALS), cancer, stroke, a metabolic disease (e.g., diabetes or obesity), a mitochondrial disorder, depression, migraines (e.g., chronic migraines), Rett syndrome, attention deficit disorder, fragile X syndrome, or traumatic brain injury (TBI)) in a subject.

In some aspects, provided herein are methods for preventing or treating a condition responsive to a ketogenic diet in a subject, comprising administering to the subject a composition comprising one or more (e.g., two or more, three or more, or four or more) compounds listed in Table 1. Also provided herein are methods of preventing or treating a condition responsive to a ketogenic diet in a subject, comprising administering to the subject a composition comprising bacteria that enrich any one or more of the gene pathways listed in Table 2.

Also provided herein are methods of treating or preventing a condition responsive to a ketogenic diet in a subject by depleting the gut microbiota of the subject and administering a composition comprising one or more compounds listed in Table 1 and/or at least one bacteria that enriches any one or more of the gene pathways listed in Table 2.

In other aspects, provided herein are methods of treating or preventing a condition responsive to a ketogenic diet in a subject by depleting the gut microbiota of the subject and administering a composition comprising bacteria that enrich any one or more of the gene pathways in Table 2. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacteria in the composition are selected from the bacteria listed in Table 4. The bacteria may be replication-competent bacteria. For example, provided herein are methods of treating or preventing a condition responsive to a ketogenic diet in a subject by depleting the gut microbiota of the subject (e.g., through antibiotics, such as broad spectrum antibodies) and administering a composition comprising at least one (at least two, at least three, at least four at least five, at least six etc.) type of bacteria listed in Table 4. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacteria in the composition are a bacteria listed in Table 4.

In some aspects, the methods comprise depleting the gut microbiota of the subject prior to administration with a composition described herein (e.g., by administering antibiotics to the subject). The subject may be a pediatric subject (e.g., the subject is under 18 years of age). The subject may be an adult (e.g. 18 years old or older). In some embodiments, the subject is no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 years of age. In some embodiments, the condition disclosed herein may be a condition in children. Thus, the condition may be a pediatric condition. The child may be less than about 1 week old. The child may be less than about 1 month old. The child may be less than about 6 months old. The child may be less than about 12 months old. The child may be less than about 2 years old. The child may be less than about 3 years old. The child may be less than about 4 years old. The child may be less than about 5 years old. The child may be less than about 6 years old. The child may be less than about 7 years old. The child may be less than about 8 years old. The child may be less than about 9 years old. The child may be less than about 10 years old. The child may be less than about 12 years old.

In some embodiments, the subject is on a diet, and the diet may be a control diet, a ketogenic diet, a high fat diet, or a low carbohydrate diet. In some embodiments, the subject is given antibiotics to deplete the subject's gut microbiota.

Also provided herein are compositions (e.g., compositions comprising any one or more of the compounds listed in Table 1 and a pharmaceutically acceptable carrier; or compositions comprising bacteria that enrich any one or more of the gene pathways in Table 2).

The composition may be formulated for oral or rectal delivery. The composition may be self-administered. The composition may be a food product. In some embodiments, the food product is a dairy product (e.g., yogurt). In some embodiments, the composition comprises probiotics. In some embodiments, the composition is self-administered. In some embodiments, the composition comprises a fecal sample (e.g., a fecal sample from a fecal bank) comprising any one or more of the bacteria of Table 4 or any bacteria that enrich any one or more of the gene pathways listed in Table 2.

### BRIEF DESCRIPTION OF THE FIGURES

FIG. 1A-FIG. 1H shows fecal transplants from epilepsy patients on the ketogenic diet confer anti-seizure effects in transplanted mice. FIG. 1A shows an experimental timeline for FMT into GF-SW mice and 6-Hz seizure testing. FIG. 1B shows seizure CC50 thresholds in response to 6-Hz seizure across each FMT cohort from 10 paired donor samples (Ordinary one-way ANOVA with Tukey's multiple comparisons test; n=14-16 mice per cohort). FIG. 1C shows averaged by cohort, mouse Pre KD vs. KD CC50 cohort thresholds across all transplanted paired sample groups (two-tailed, unpaired Welch's t-test, n refers to the number of Pre KD or Post KD FMT cohorts, n=10)). FIG. 1D shows latency to exploration of all mice that underwent 6-Hz seizure testing (n refers to the number of SW mice tested n=141 (Pre), 146 (Post)) FIG. 1E shows average remission seizure length across all FMT groups during 6-Hz testing. (two-tailed, unpaired Welch's t-test, n refers to the number of remission seizures; n=54 (Pre); 40 (Post)) FIG. 1F shows average remission current of remission seizures observed across all patient groups during 6-Hz testing. (two-tailed, unpaired Welch's t-test, n refers to the number of remission seizures; n=54 (Pre); 40 (Post)). FIG. 1G shows individually plotted remission seizure lengths observed at the current induced during 6-Hz seizure testing. (n refers to the number of remission seizures; n=54 (Pre); 40 (Post)). FIG. 1H shows the percentage of mice across all cohorts with an observed behavior associated with 6-Hz seizure testing. Data is displayed as mean+/−SEM, unless otherwise noted. ***P<0.001, ****P<0.0001; FMT, fecal microbiome transplant; GF, germ-free mice; SW, swiss webster mice; KD, ketogenic diet.

FIG. 2A-FIG. 2D shows fidelity of human microbiota transplantation into mice: taxonomic data. FIG. 2A shows principal coordinate analysis of weighted UniFrac distances for donor Pre KD and donor Post KD fecal samples. (n=10 Pre KD; 10 Post KD) FIG. 2B shows principal coordinate analysis of unweighted UniFrac distances for donor Pre KD and donor Post KD fecal samples (n=10 Pre KD; 10 Post KD). FIG. 2C shows comparison of Shannon Index alpha-diversity between all donor and all recipient 16s rDNA sequencing samples (two-tailed, paired Student's t-test, n=2 donor samples; 10 recipient cages/groups). FIG. 2D shows principal coordinate analysis of weighted UniFrac distances per Donor patient pair and corresponding recipient samples (n=1 donor Pre KD; 1 donor Post KD; 5 recipient Pre KD cages/groups; 5 recipient Post KD cages/groups). Data is displayed as mean+/−SEM, unless otherwise noted. **P<0.01

FIG. 3A-FIG. 3C shows fidelity of human microbiome transplantation into mice: functional metagenomic data. FIG. 3A shows principal component analysis comparing donor Pre KD and Post KD metagenomes (n=10 Pre KD; 10 Post KD). FIG. 3B shows principal component analysis comparing recipient Pre KD and Post KD metagenomes (n=10 Pre KD; 10 Post KD). FIG. 3C shows averaged observed OTUs from 16S rDNA sequencing of Donor Pre KD and Donor Post KD stool samples and corresponding recipient mice transplanted with donor samples' fecal pellets (n=5 recipient cages/groups, 1 donor sample (Pre and Post)).

FIGS. 4A-FIG. 4C shows influence of recipient mouse sex on seizure susceptibility in response to human microbiota transplantation. FIG. 4A shows experimental timeline for FMT into GF-SW mice and 6-Hz seizure testing. FIG. 4B shows seizure CC50 thresholds in response to 6-Hz seizure testing 4 days post gavage in cohorts of SW mice transplanted with Patient 3 Pre ketogenic diet (KD) initiation or 1-month Post KD initiation human fecal sample (Ordinary one-way ANOVA with Tukey's multiple comparisons test; n refers to the size of the cohort tested with n=15 (Pre (f)), 14 (Post (f)); 14 (Pre (m)), 14 (Post (m)). FIG. 4C shows latency to exploration after initial 6 Hz testing shock (n refers to the size of the cohort tested with n=15 (Pre (f)), 14 (Post (f)); 14 (Pre (m)), 14 (Post (m)). Data is displayed as mean+/−SEM. ****P<0.0001; n.s., no significance; f, female; m. male; FMT, fecal microbiome transplant; GF, germ-free mice; SW, swiss webster mice; KD, ketogenic diet.

FIG. 5A-FIG. 5D shows antibiotic treatment abrogates the seizure protective effects of transplantation with the clinical KD-associated human gut microbiota. FIG. 5A shows an experimental timeline for FMT and ABX depletion schedule with 6-Hz seizure testing. FIG. 5B shows 16s rDNA Gene Abundance by Ct value from fecal pellets. Collected once daily (n refers to the number of cages collects from, one mouse pellet per cage, n=3). Error bars represent SD. FIG. 5C shows seizure CC50 thresholds in response ABX depletion, 12 days post gavage. (Ordinary one-way ANOVA with Tukey's multiple comparisons test; n refers to the size of the cohort tested with n=14 (Post+Veh); 14 (Post+Abx); 14 (Post); 14 (Pre)). FIG. 5D shows latency to exploration of all mice that underwent 6-Hz seizure testing (n refers to the number of SW mice tested n=14 (Post+Veh); 14 (Post+Abx); 14 (Post); 14 (Pre)). Data is displayed as mean+/−SEM unless otherwise noted. ****P<0.0001; FMT, fecal microbiome transplant; GF, germ-free mice; SW, swiss webster mice; KD, ketogenic diet.

FIG. 6A-FIG. 6C shows sterile filtration prevents the seizure protective effects of the clinical KD-associated human gut microbiota. FIG. 6A shows an experimental timeline for FMT into GF-SW mice and 6-Hz seizure testing. FIG. 6B shows a seizure CC50 thresholds in response to 6-Hz seizure testing 4 days post gavage in cohorts of SW mice transplanted with Patient 2 Pre ketogenic diet (KD) initiation or 1-month Post KD initiation human fecal sample. Error bars plotted as mean+/−Standard Deviation. (Ordinary one-way ANOVA with Tukey's multiple comparisons test; n refers to the size of the cohort tested with n=14 (Pre), 14 (Post); 13 (Post+Filter)). FIG. 6C shows a latency to exploration of male and female mice after initial 6 Hz testing shock (n refers to the number of male SW mice tested n=14 (Pre), 14 (Post); 13 (Post+Filter)). Data is displayed as mean+/−SEM. ****P<0.0001; FMT, fecal microbiome transplant; GF, germ-free mice; SW, swiss webster mice; KD, ketogenic diet

FIG. 7A-FIG. 7G shows the clinical KD-associated human microbiome exhibits functional alterations that are recapitulated in seizure-protected recipient mice. FIG. 7A shows a heatmap of metagenome MetaCyc pathways significantly associated with Pre or Post states by Maaslin2 analysis (simple linear statistical model; n=10 Donor Pre, 10 Donor Post, 10 Recipient Pre, 10 Recipient Post). FIG. 7B shows a metaCyc pathways of interest from metagenomic analysis significantly associated with Pre KD or Post KD state across corresponding human donor and mouse recipient groups (simple linear statistical model; n=10 Donor Pre, 10 Donor Post, 10 Recipient Pre, 10 Recipient Post). FIG. 7C shows relative concentrations of BHB in Pre KD vs Post KD human donor fecal (left), mouse recipient fecal (middle), and mouse recipient serum (right) samples (two-sided Student's t-test; n=10 (Pre); 10 (Post)). FIG. 7D shows a relative concentrations of glucose in Pre KD vs Post KD human donor fecal (left), mouse recipient fecal (middle), and mouse recipient serum (right) samples (two-sided Student's t-test; n=10 (Pre); 10 (Post)). FIG. 7E shows a fold-change in lipid abundances between Pre KD and Post KD states in human donor fecal, mouse recipient fecal, and mouse recipient serum (matched pairs Student's t-test; n=10 (Pre); 10 (Post)). FIG. 7F shows a fold-change in tryptophan related metabolite abundances between Pre KD and Post KD states in human donor fecal, mouse recipient fecal, and mouse recipient serum (matched pairs Student's t-test; n=10 (Pre); 10 (Post)). FIG. 7G shows a fold-change in bile acid abundances between Pre KD and Post KD states in human donor fecal, mouse recipient fecal, and mouse recipient serum (matched pairs Student's t-test; n=10 (Pre); 10 (Post)). Data is displayed as mean+/−SEM, unless otherwise noted. +0.05<P<0.1; *P<0.05; KD, ketogenic diet.

FIG. 8A-FIG. 8E shows taxonomic analysis of the clinical KD-associated human microbiota and transplanted recipient mice relative to pre-KD controls. FIG. 8A shows principal coordinate analysis of unweighted UniFrac distances from 16S rDNA sequencing of donor and recipient Pre KD and Post KD fecal samples (n=10 Donor Pre; 10 Donor Post; 50 Recipient Pre; 50 Recipient Post). FIG. 8B shows the principal coordinate analysis of weighted UniFrac distances from 16S rDNA sequencing of donor and recipient Pre KD and Post KD fecal samples (n=10 Donor Pre; 10 Donor Post; 50 Recipient Pre; 50 Recipient Post). FIG. 8C shows an average relative abundance of the most abundant species from all recipient pre or all recipient post samples. The legend denotes abundant species >0.5% relative abundance. (n=50 recipient cages/groups (Pre and Post)). FIG. 8D shows an average relative abundance of rare species from all recipient pre or all recipient post samples. The legend denotes rare species of between 0.05%-0.5% relative abundance (n=50 recipient cages/groups (Pre and Post)). FIG. 8E shows ANCOM statistical analysis (left) highlights g_Dorea; s_as significantly altered relative abundance between Pre KD and Post KD (right) . . . (n=50 recipient cages/groups (Pre and Post))**P<0.01; KD, ketogenic diet.

FIG. 9A-FIG. 9D shows additional metabolomic data. FIG. 9A shows random forest analysis on metabolomic datasets from human fecal (left), mouse fecal (middle) and mouse serum (right) for Pre KD vs Post KD respective predictive accuracies of 65%. 65%, and 55%. FIG. 9B shows principal component analysis comparing donor Pre KD and Post KD fecal metabolomes (n=10 Pre KD; 10 Post KD). FIG. 9C shows principal component analysis comparing recipient Pre KD and Post KD fecal metabolomes (n=10 Pre KD; 10 Post KD). FIG. 9D shows principal component analysis comparing recipient Pre KD and Post KD serum metabolomes (n=10 Pre KD; 10 Post KD).

FIG. 10 shows taxonomic composition of human pediatric epilepsy microbiome before and after the clinical ketogenic diet.

FIG. 11A-F shows associations between the clinical KD and alterations in the composition and functional potential of the gut microbiome of children with refractory epilepsy. FIG. 11A shows a rarefaction curve (left) and Shannon Index (right) for alpha-diversity of matched donor pre-KD and post-KD fecal microbiota samples. Repeated measures ANOVA (left), two-tailed, paired Student's t-test (right), n=10 patient samples per group. FIG. 11B shows principal coordinates analysis of unweighted (left) and weighted (right) UniFrac distances from 16S rRNA gene sequencing of donor pre-KD and post-KD fecal microbiota samples (n=10 patient samples per group). FIG. 11C shows W (effect size) plotted against center-log ratio for ANCOM differential abundance testing of each microbial species abundance comparing Donor Pre-KD and Donor Post-KD (n=10 Donor Pre-KD; 10 Donor Post-KD). FIG. 11D shows relative abundance of (left) MetaCyc superclass metagenome functional pathways and (right) difference in relative abundance of the top 26 most abundant pathways, accounting for >95% of relative abundance across donor pre-KD and post-KD fecal microbiota samples. (n=10 patient samples per group). FIG. 11E shows the number of observed MetaCyc functional pathways in donor pre-KD and post-KD fecal microbiota samples (two-tailed, Wilcoxon matched-pairs signed rank test, n=10 patient samples per group). FIG. 11F shows heatmap of MetaCyc functional pathways differentially abundant in donor post-KD relative to pre-KD by Maaslin2 analysis with cutoff at p<0.1. Donor pre-KD (n=10), donor post-KD (n=10). Data is displayed as mean+SEM, unless otherwise noted. **p<0.01. KD=ketogenic diet, n.s.=not statistically significant.

FIG. 12A-12E shows transplantation of the clinical KD-associated gut microbiota from pediatric epilepsy patients confers seizure protection in mice. FIG. 12A shows experimental design for transplantation of human donor fecal microbiota samples into germ-free Swiss Webster (GF-SW) mice for 6-Hz psychomotor seizure testing. FIG. 12B shows seizure thresholds for replicate mice transplanted with human microbiota from paired donor pre-KD and post-KD samples (One-way ANOVA with Tukey's, n=14-16 mice per patient sample, with denoting statistical differences when considering within-patient recipient mice as technical replicates). FIG. 12C shows the average seizure thresholds of recipient mouse cohorts per patient donor sample. (Two-tailed, unpaired Welch's t-test. n=10 patient samples per group). FIG. 12D shows latency to exploration for all transplanted mice that underwent 6-Hz psychomotor seizure testing. (n=XXX-YYY per group). FIG. 12E shows average current (mA) at which remission seizures were observed. (Two-tailed, unpaired Welch's t-test. n=10 patient samples per group) Data is displayed as mean+SEM, unless otherwise noted. ####p<0.0001 (within-patient mouse recipients), ***p<0.001, ****p<0.0001

FIG. 13A-C shows taxonomic fidelity of human microbiota transplantation into mice. FIG. 13A shows principal coordinates analysis of weighted UniFrac distances from 16S rRNA gene sequencing of fecal samples from matched human donors and mouse recipients (for each graph: n=1 patient, 5 cages of recipient mice per pre-KD vs. post-KD condition). FIG. 13B shows the Shannon index alpha-diversity of fecal microbiota from human donor samples (left) and matched mouse recipients (right) (two-tailed, paired Student's t-test. n=10 per group). FIG. 13C shows the Shannon index alpha-diversity of fecal microbiota from all human donor samples and all matched mouse recipients (two-tailed, paired Student's t-test. n=20 (pre-KD+post-KD) per condition). Data is displayed as mean #SEM, unless otherwise noted. ***p<0.001, n.s.=not statistically significant.

FIG. 14A-D shows an antibiotic treatment abrogates the seizure protective effects of transplantation with the clinical KD-associated human gut microbiome, Related to FIG. 12. FIG. 14A shows the experimental design for transplantation of human donor fecal microbiota samples into germ-free (GF) mice, followed by 5 days of oral antibiotic (Abx) or vehicle (Veh) treatment, and then 6-Hz psychomotor seizure testing. FIG. 14B shows the bacterial loads as measured by quantitative PCR of the 16S rRNA gene from fecal pellets collected once daily before and during Abx or Veh treatment (Two-way ANOVA with Sidak, n=3 cages per condition). FIG. 14C shows seizure thresholds for mice transplanted with the post-KD human microbiota and treated with Abx or Veh. Reference lines denote seizure thresholds for mice transplanted with the same post-KD human microbiota without further Abx or Veh treatment, from data in FIG. 12. (One-way ANOVA with Tukey's, n=14 mice per condition, with #denoting statistical differences when considering within-patient recipient mice as technical replicates). FIG. 14D shows the latency to exploration for mice that underwent 6-Hz psychomotor seizure testing (n=14 mice per condition). Data is displayed as mean+SEM, unless otherwise noted. ****p<0.0001. ####p<0.0001 (for within-patient mouse recipients).

FIG. 15A-C shows sterile filtration prevents the seizure protective effects of transplantation with the clinical KD-associated human gut microbiome, Related to FIG. 12. FIG. 15A shows experimental design for administration of human donor fecal microbiota or fecal filtrate samples to germ-free (GF) mice, followed by 6-Hz seizure testing 4 days later. FIG. 15B shows seizure thresholds for mice treated with sterile filtered fecal samples relative unfiltered fecal microbiota from the post-KD human microbiota relative to matched pre-KD controls. Data for mice transplanted with pre-KD and post-KD fecal microbiota are as in FIG. 12. (One-way ANOVA with Tukey's, n=14 mice per condition, with #denoting statistical differences when considering within-patient recipient mice as technical replicates). FIG. 15C shows latency to exploration for mice that underwent 6-Hz psychomotor seizure testing (n=14 mice per condition). Data for mice transplanted with pre-KD and post-KD fecal microbiota are as in FIG. 12. Data is displayed as mean±SEM, unless otherwise noted. ####p<0.0001 (for within-patient mouse recipients).

FIG. 16A-C shows small molecules from the clinical KD-associated human gut microbiome confer acute seizure protection, Related to FIG. 12. FIG. 16A shows experimental design for administration of human donor fecal filtrate samples to germ-free (GF) mice, followed by 6-Hz seizure testing 2 hours later. FIG. 16B shows seizure thresholds for mice treated with sterile filtered fecal samples from the post-KD human microbiota relative to matched pre-KD control. Reference lines denote seizure thresholds for mice transplanted with the same post-KD human microbiota, from data in FIG. 12. (One-way ANOVA with Tukey's, n=14 mice per condition, with #denoting statistical differences when considering within-patient recipient mice as technical replicates). FIG. 16C shows latency to exploration for mice that underwent 6-Hz psychomotor seizure testing (n=14 mice per condition). Data is displayed as mean±SEM, unless otherwise noted. ####p<0.0001 (for within-patient mouse recipients).

FIG. 17A-F shows the clinical KD-associated human microbiome exhibits functional alterations that are recapitulated in seizure-protected recipient mice. FIG. 17A shows microbial functional pathways differentially abundant in post-KD samples relative to pre-KD controls for either donor fecal samples or recipient fecal samples. Red font denotes pathways that are commonly differentially abundant in the same direction in both donor and recipient samples. (n=10 donor patient samples per condition, n=50 recipient mice per condition, reflecting 5 recipient mice per donor patient sample). FIG. 17B shows microbial functional pathways with shared differential abundance in post-KD donor and recipient fecal microbiomes relative to pre-KD donor and recipient controls. (n=10 donor patient samples per condition, n=50 recipient mice per condition, reflecting 5 recipient mice per donor patient sample). FIG. 17C shows beta-hydroxybutyrate (BHBA) and glucose levels in human donor pre-KD and post-KD feces and serum (left) and mouse recipient pre-KD and post-KD feces and serum (right). (Two-sided, paired Student's t-test, n=10 donor patient samples per condition, where each mouse value reflects an average of 5 recipient mice per donor patient sample). FIG. 17D shows fold-change in lipid abundances between pre KD and post KD states in human donor fecal, mouse recipient fecal, and mouse recipient serum (matched pairs Student's t-test; n=10 (Pre); 10 (Post)). FIG. 17E shows fold-change in tryptophan related metabolite abundances between pre KD and post KD states in human donor fecal, mouse recipient fecal, and mouse recipient serum (matched pairs Student's t-test; n=10 (pre); 10 (post)). FIG. 17F shows fold-change in bile acid abundances between pre KD and post KD states in human donor fecal, mouse recipient fecal, and mouse recipient serum (matched pairs Student's t-test; n=10 (pre); 10 (post)).

FIG. 18A-18C shows characterization of short-chain fatty acid levels in human donor and mouse recipient pre-KD and post-KD fecal and serum samples, Related to FIG. 12. FIG. 18A shows short-chain fatty acid levels in human donor pre-KD and post-KD fecal samples. (Two-sided, paired Student's t-test, n=10 donor patient samples per condition, where each mouse value reflects an average of 5 recipient mice per donor patient sample).

FIG. 18B shows short-chain fatty acid levels in mouse recipient pre-KD and post-KD fecal samples. (Two-sided, paired Student's t-test, n=10 donor patient samples per condition, where each mouse value reflects an average of 5 recipient mice per donor patient sample). FIG. 18C shows short-chain fatty acid levels in mouse recipient pre-KD and post-KD serum samples. (Two-sided, paired Student's t test, n=10 donor patient samples per condition, where each mouse value reflects an average of 5 recipient mice per donor patient sample).

FIG. 19A-D shows human donor and mouse recipient metabolite pathway enrichment analysis. FIG. 19A shows volcano plot of enriched metabolites in human donor post-KD compared to pre-KD fecal samples, with minimum FC>2.0 and p-value<0.1. FIG. 19B shows metabolite set enrichment analysis in human donor post-KD fecal samples from the identified subset of metabolites from (FIG. 19A). FIG. 19C shows volcano plot of enriched metabolites in mouse recipient post-KD compared to pre-KD fecal samples, with minimum FC>2.0 and p-value<0.1. FIG. 19D shows metabolite set enrichment analysis in mouse recipient post-KD fecal samples from the identified subset of metabolites from FIG. 19C.

FIG. 20A-B shows recipient mouse pre-KD and post-KD hippocampal and frontal cortex transcriptomics. FIG. 20A shows GO enrichment of differentially expressed genes in recipient mouse post-KD compared to pre-KD frontal cortex tissue samples (cutoff, q-value<0.1). FIG. 20A also shows a heatmap of top differentially expressed genes in recipient mouse post-KD compared to pre-KD frontal cortex tissue samples with q-value<0.05 and fold-changes >1.5. FIG. 20B shows GO enrichment of differentially expressed genes in recipient mouse post-KD compared to pre-KD hippocampal tissue samples (cutoff, q-value<0.1). FIG. 20B also shows a heatmap of top differentially expressed genes in recipient mouse post-KD compared to pre-KD hippocampal tissue samples with q-value<0.05 and fold-changes >1.5.

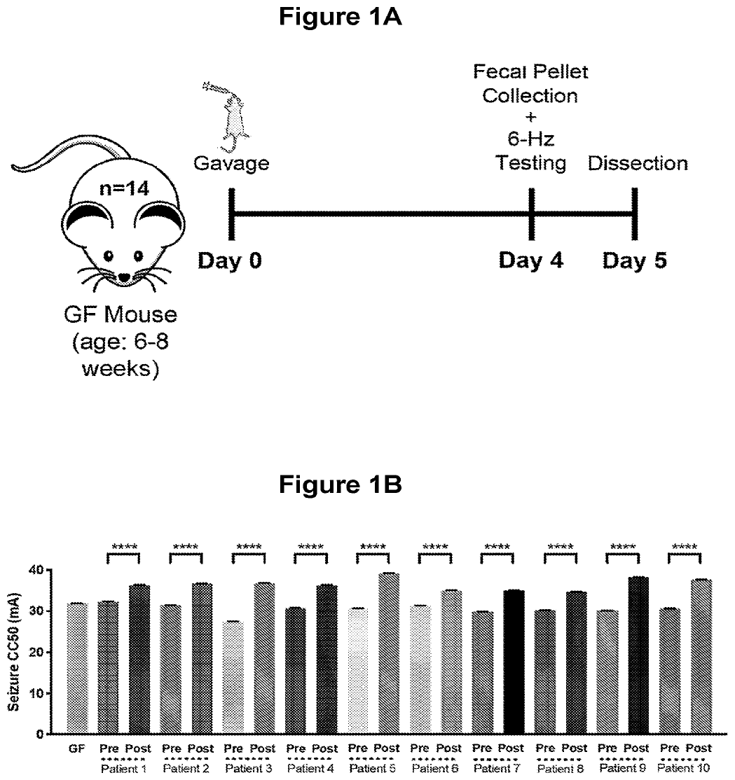

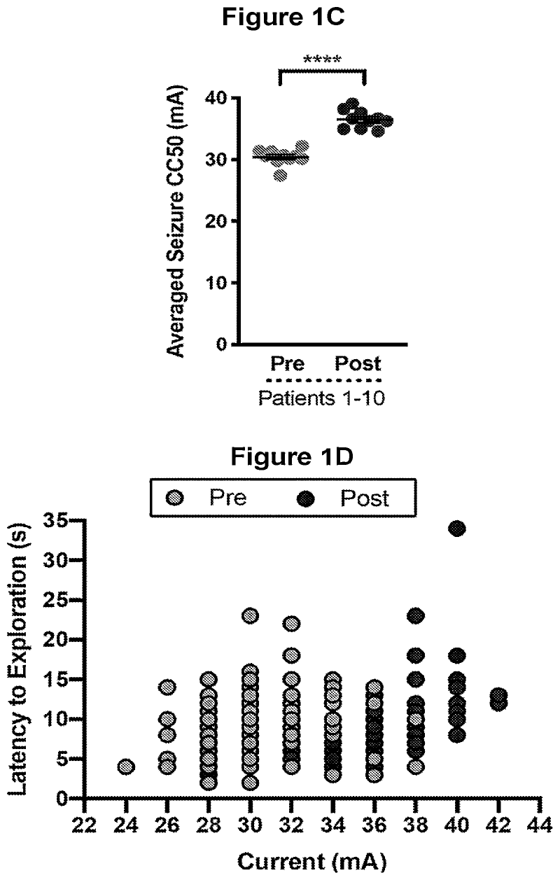

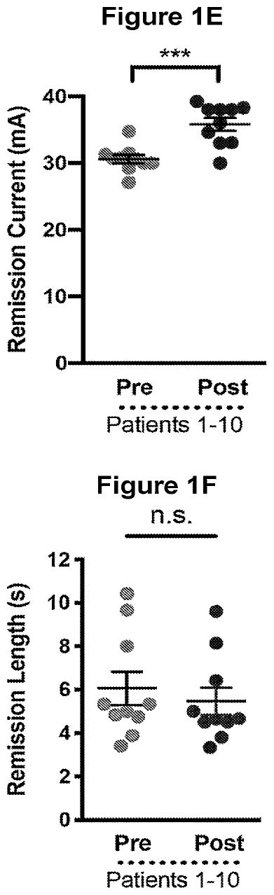

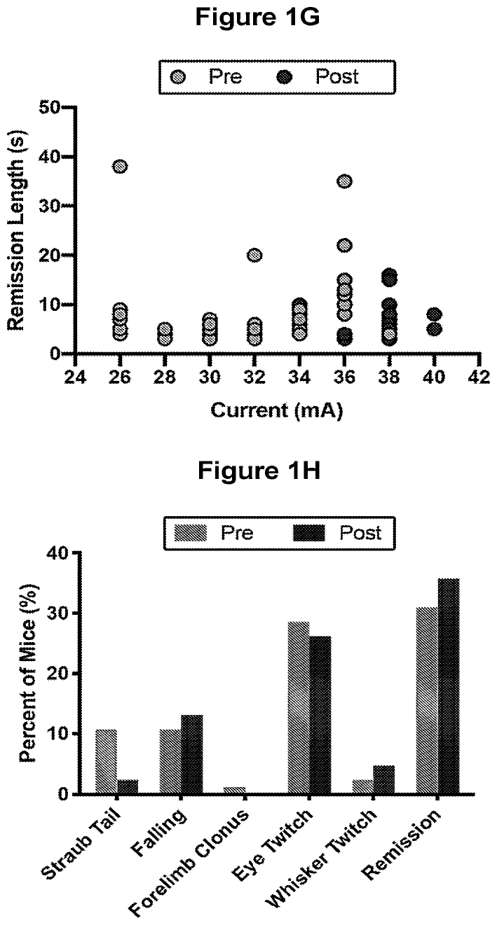

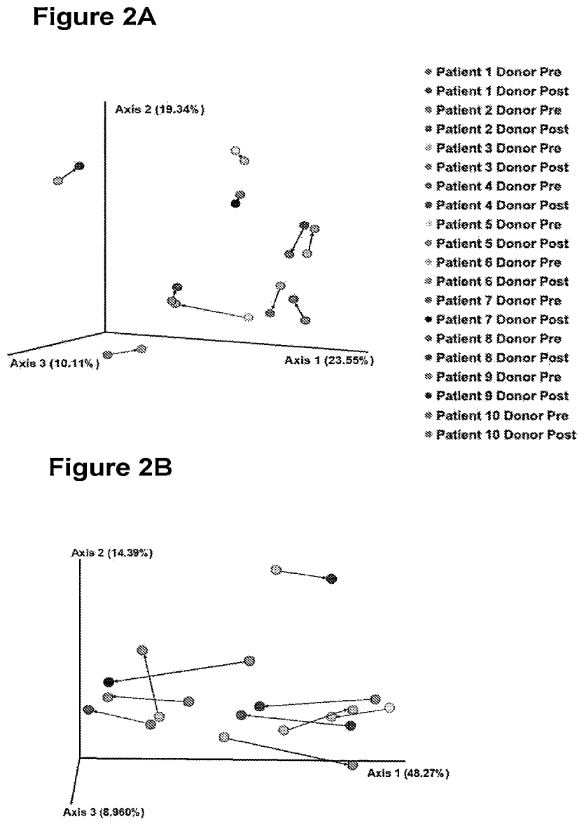

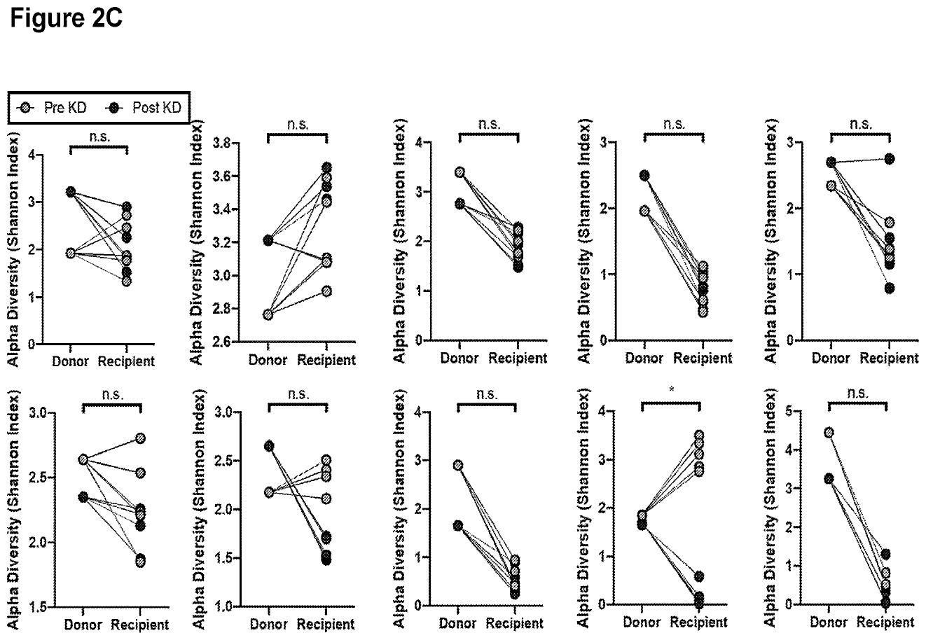

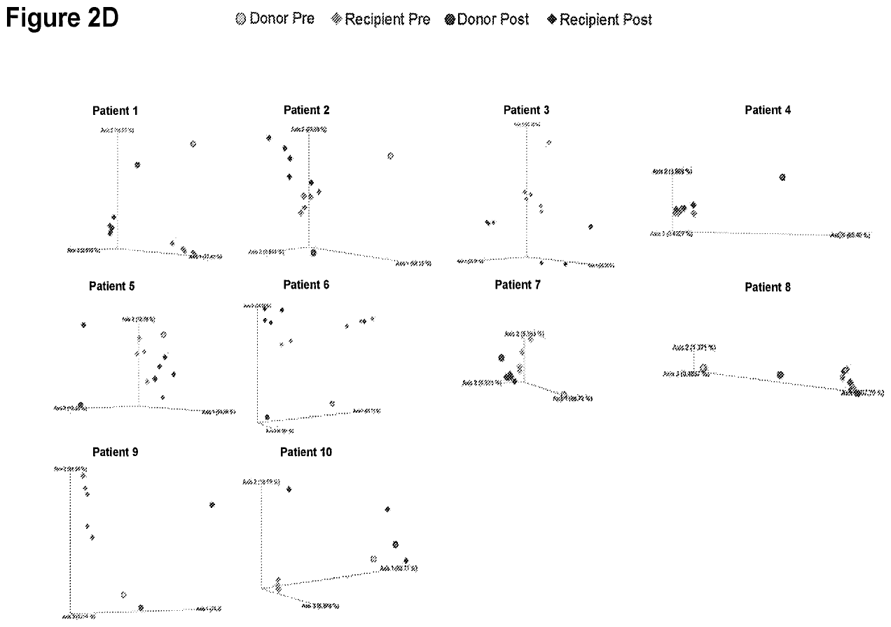

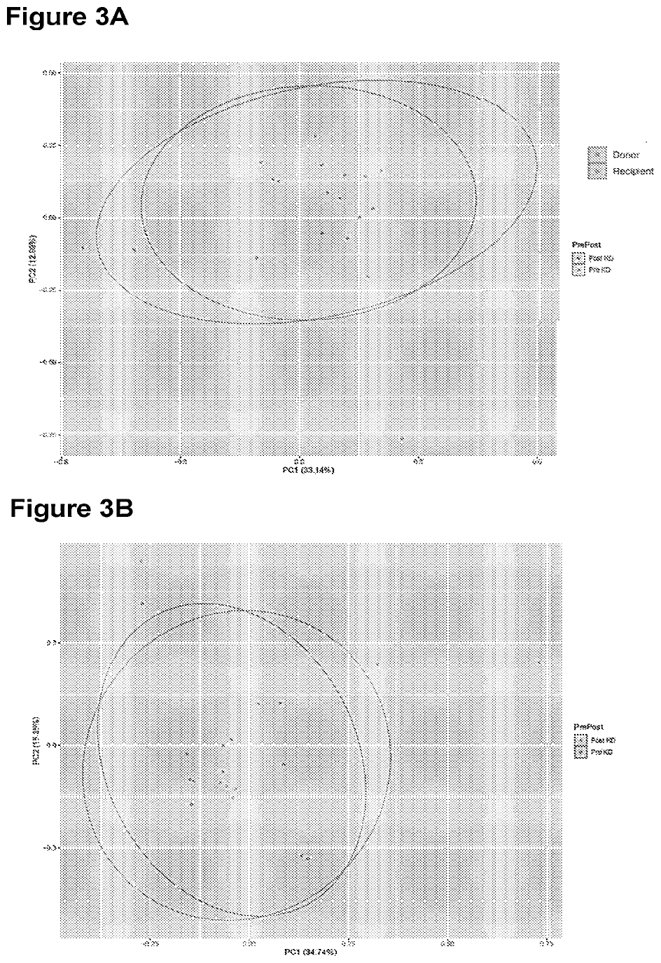

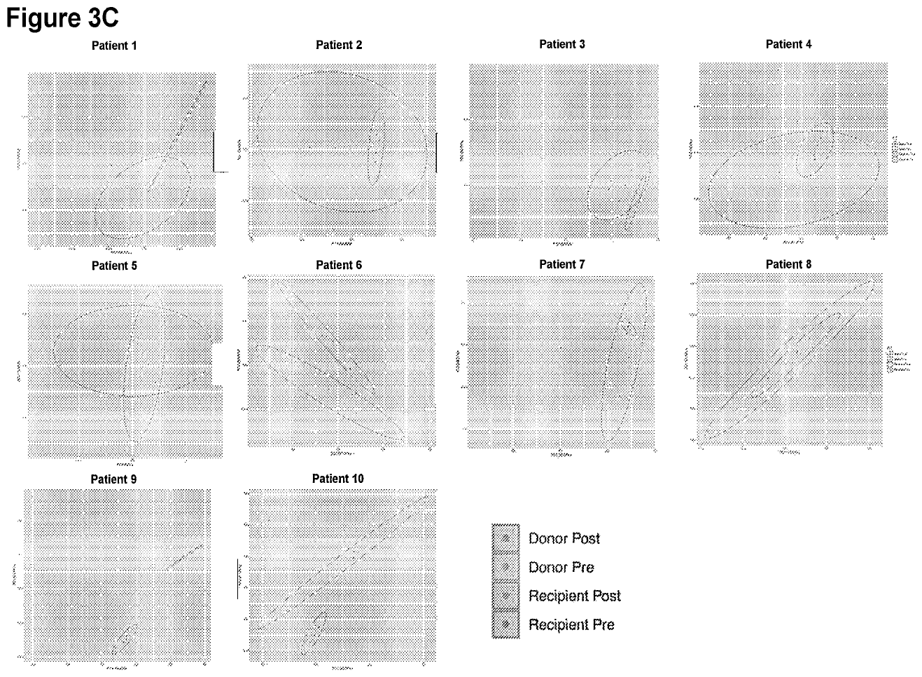

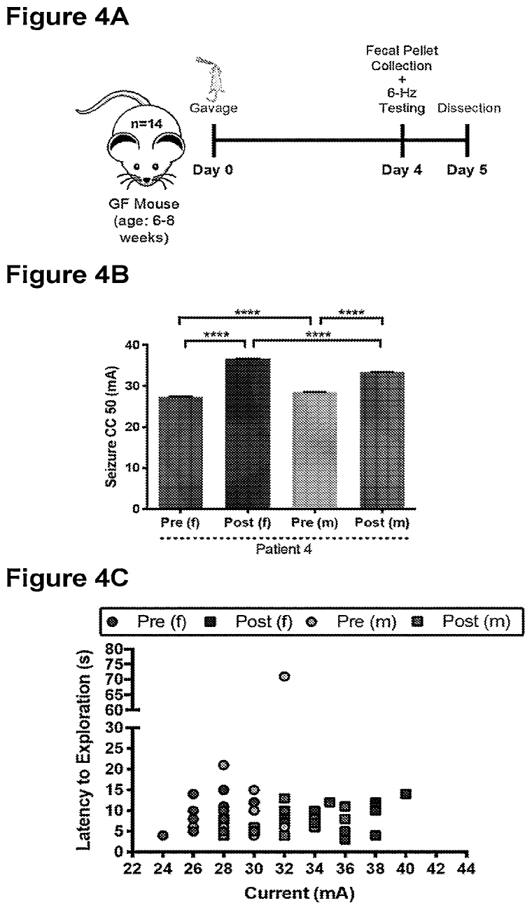

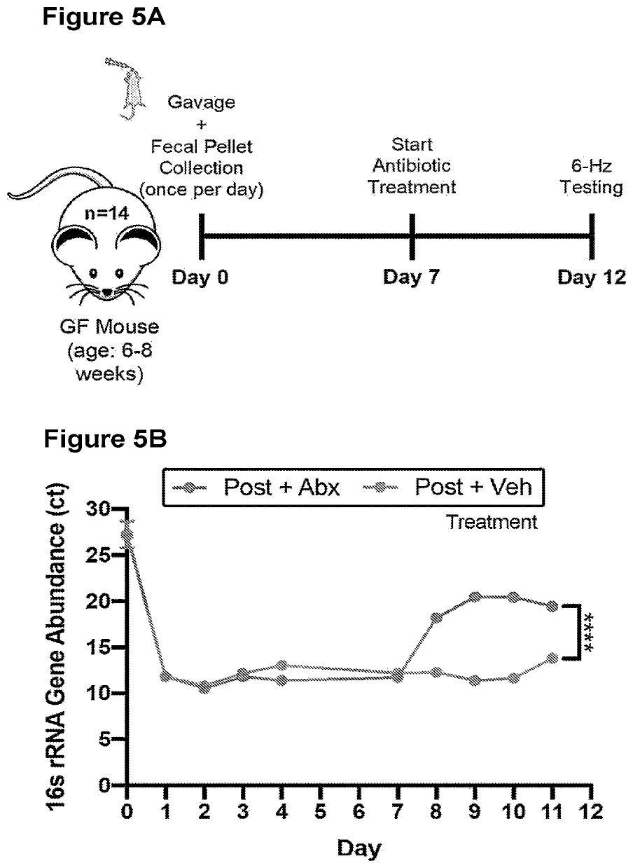

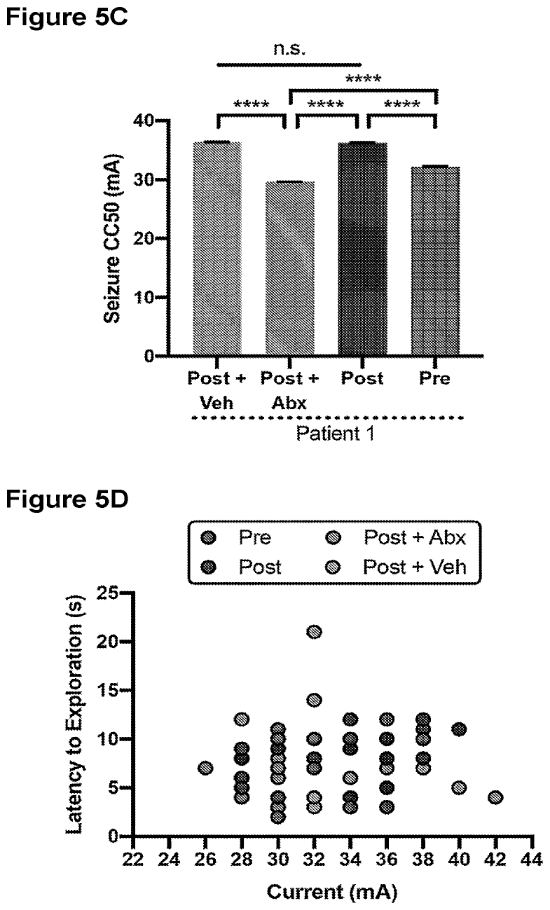

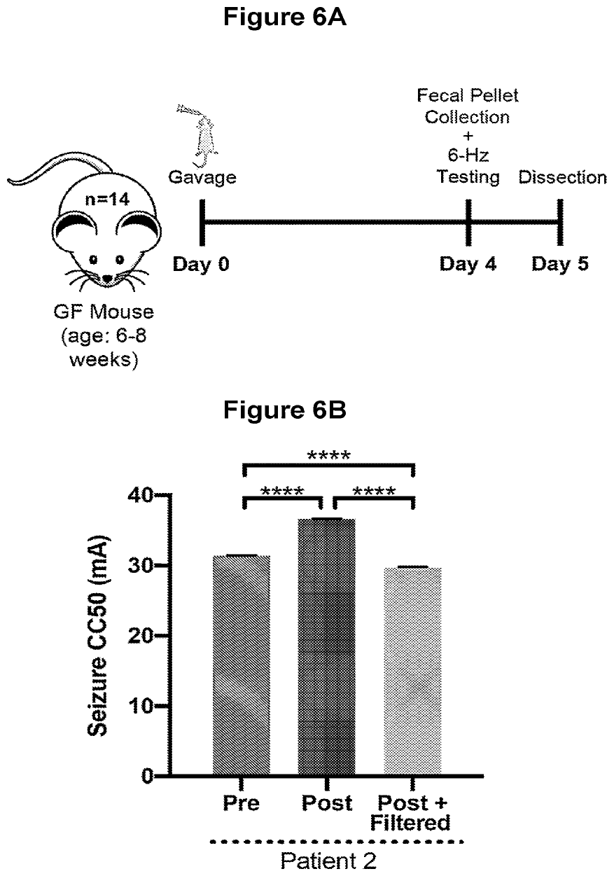

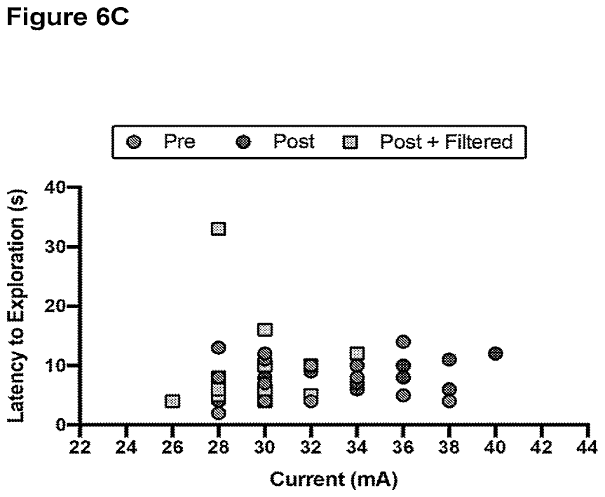

### DETAILED DESCRIPTION

Provided herein are methods and compositions for mimicking the effects of a ketogenic diet by administering compositions provided herein.

In some aspects, provided herein are methods for preventing or treating a condition responsive to a ketogenic diet in a subject, comprising administering to the subject a composition comprising one or more compounds listed in Table 1. Also provided herein are methods of preventing or treating a condition responsive to a ketogenic diet in a subject, comprising administering to the subject a composition comprising bacteria that enrich any one or more of the gene pathways listed in Table 2.

Also provided herein are methods of treating or preventing a condition responsive to a ketogenic diet in a subject by depleting the gut microbiota of the subject and administering a composition comprising one or more compounds listed in Table 1.

In other aspects, provided herein are methods of treating or preventing a condition responsive to a ketogenic diet in a subject by depleting the gut microbiota of the subject and administering a composition comprising bacteria that enrich any one or more of the gene pathways listed in Table 2. In some embodiments, at least 10%, at least 20%, at least 30%, at least 40%, at least 50%, at least 60%, at least 70%, at least 80%, at least 90%, at least 95%, at least 99%, or 100% of the bacteria in the composition are selected from the bacteria listed in Table 4. The bacteria may be replication-competent bacteria.

In some aspects, the methods comprise depleting the gut microbiota of the subject prior to administration with a composition described herein (e.g., by administering antibiotics to the subject).

In some embodiments, the subject is on a diet, and the diet may be a control diet, a ketogenic diet, a high fat diet, or a low carbohydrate diet. In some embodiments, the subject is given antibiotics to deplete the subject's gut microbiota.

Also provided herein are compositions (e.g., compositions comprising any one of the compounds listed in Table 1 and a pharmaceutically acceptable carrier; or compositions comprising bacteria that enrich any one or more of the gene pathways listed in Table 2).

In certain embodiments, the methods and compositions are for the treatment or prevention of seizures in a subject (e.g., a subject with a neurodevelopmental disorder, such as an autism spectrum disorder, Rett syndrome, fragile X, attention deficit disorder (ADD), attention-deficit/hyperactivity disorder (ADHD), refractory epilepsy, such as pediatric refractory epilepsy, and/or non-refractory epilepsy). In other embodiments, the methods and compositions are for preventing or treating a condition (e.g., autism spectrum disorder, epilepsy, seizures, Alzheimer's disease, Huntington's disease, Parkinson's disease, amyotrophic lateral sclerosis (ALS), cancer, stroke, a metabolic disease (e.g., obesity or diabetes), a mitochondrial disorder, depression, migraines (e.g., chronic migraines), Rett syndrome, attention deficit disorder, fragile X syndrome, or traumatic brain injury (TBI)) in a subject.

### Definitions

As used herein in the specification, “a” or “an” may mean one or more. As used herein in the claim(s), when used in conjunction with the word “comprising”, the words “a” or “an” may mean one or more than one. As used herein “another” may mean at least a second or more.

The phrase “pharmaceutically-acceptable carrier” as used herein means a pharmaceutically-acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “preventing” is art-recognized, and when used in relation to a condition, such as a local recurrence, is well understood in the art, and includes administration of a composition which reduces the frequency of, or delays the onset of, symptoms of a medical condition in a subject relative to a subject which does not receive the composition. Thus, prevention of seizures includes, for example, reducing the number of seizures in a population of patients receiving a prophylactic treatment relative to an untreated control population, e.g., by a statistically and/or clinically significant amount.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “subject” refers to a mammal, including, but not limited to, a human or non-human mammal, such as a bovine, equine, canine, ovine, or feline.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

### Therapeutic Methods

The disclosure herein, relates, in part, to the discovery that fecal transplant from a patient given a ketogenic diet can protect against, for example, pediatric seizures. In addition, Applicant has discovered that specific metabolites and bacteria with in the gut microbiome can protect and treat disorders treated with a ketogenic diet.

A ketogenic diet (KD) induces substantial changes in the gut microbiome, and that enriching KD-associated bacteria via probiotic administration, fecal transplant, or selective microbial reconstitution of the native microbiome mimics the beneficial effects of the KD. Provided herein are methods and compositions that can replace the KD diet in the treatment or prevention of a condition as described. The methods and compositions described herein can be used separately or in conjunction with the KD diet in the treatment or prevention of a condition described herein.

In some embodiments, the subject is on a diet, and the diet may be a control diet, a ketogenic diet, a high fat diet, or a low carbohydrate diet. In some embodiments, the subject is given antibiotics to deplete the subject's gut microbiota.

In certain embodiments, the methods treat or prevent seizures in a subject. In some aspects, provided herein are methods for preventing or treating a condition responsive to a ketogenic diet in a subject, comprising administering to the subject a composition comprising a compound listed in Table 1. The composition may comprise at least one, at least two, at least three, at least four, at least five, at least six at least seven, at least eight, at least nine, at least ten, at least fifteen, or at least twenty of the compounds listed in Table 1. The composition may comprise at least at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 of the compounds listed in Table 1. Also provided herein are methods of preventing or treating a condition responsive to a ketogenic diet in a subject, comprising administering to the subject a composition comprising bacteria that enrich any one of the gene pathways listed in Table 2. The bacteria may enrich for at least one, at least two, at least three, at least four, at least five, at least six at least seven, at least eight, at least nine, or at least ten of the gene pathways listed in Table 2.

Also provided herein are methods of treating or preventing a condition responsive to a ketogenic diet in a subject by depleting the gut microbiota of the subject and administering a composition comprising a compound listed in Table 1. The composition may comprise at least one, at least two, at least three, at least four, at least five, at least six at least seven, at least eight, at least nine, at least ten, at least fifteen, or at least twenty of the compounds listed in Table 1.

In other aspects, provided herein are methods of treating or preventing a condition responsive to a ketogenic diet in a subject by depleting the gut microbiota of the subject and administering a composition comprising bacteria that enrich any one of the gene pathways in Table 2. The bacteria may enrich for at least one, at least two, at least three, at least four, at least five, at least six at least seven, at least eight, at least nine, or at least ten of the gene pathways listed in Table 2. In some embodiments, the compositions comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 of the bacteria listed in Table 4. In some embodiments, at least 10%, at least 20%, at least 30%, at least 40%, at least 50%, at least 60%, at least 70%, at least 80%, at least 90%, at least 95%, at least 99%, or 100% of the bacteria in the composition are selected from the bacteria listed in Table 4. For example, in some embodiments, at least 10%, at least 20%, at least 30%, at least 40%, at least 50%, at least 60%, at least 70%, at least 80%, at least 90%, at least 95%, at least 99%, or 100% of the bacteria in the composition are at least one (e.g., at least two, at least three, at least four, at least five, at least six, or at least seven) of the bacteria listed in Table 4. The composition may comprise at least one, at least two, at least three, at least four, at least five, at least six at least seven, at least eight, at least nine, at least ten, at least fifteen, or at least twenty of the bacteria listed in Table 4. Any combination of the bacteria listed in Table 4 may be include in the composition. The bacteria may be replication-competent bacteria.

In some aspects, the methods comprise depleting the gut microbiota of the subject prior to administration with a composition described herein (e.g., by administering antibiotics to the subject).

In some embodiments, the subject has epilepsy (e.g., pediatric epilepsy; refractory or non-refractory epilepsy). The disclosed epilepsy disorder may be benign Rolandic epilepsy, frontal lobe epilepsy, infantile spasms, juvenile myoclonic epilepsy (JME), juvenile absence epilepsy, childhood absence epilepsy (e.g. pyknolepsy), febrile seizures, progressive myoclonus epilepsy of Lafora, Lennox-Gastaut syndrome, Landau-Kleffner syndrome, Dravet syndrome, Generalized Epilepsy with Febrile Seizures (GEFS+), Severe Myoclonic Epilepsy of Infancy (SMEI), Benign Neonatal Familial Convulsions (BFNC), West Syndrome, Ohtahara Syndrome, early myoclonic encephalopathies, migrating partial epilepsy, infantile epileptic encephalopathies, Tuberous Sclerosis Complex (TSC), focal cortical dysplasia, Type I Lissencephaly, Miller-Dieker Syndrome, Angelman's syndrome, Fragile X syndrome, epilepsy in autism spectrum disorders, subcortical band heterotopia, Walker-Warburg syndrome, Alzheimer's disease, posttraumatic epilepsy, progressive myoclonus epilepsies, reflex epilepsy, Rasmussen's syndrome, temporal lobe epilepsy, limbic epilepsy, status epilepticus, abdominal epilepsy, massive bilateral myoclonus, catamenial epilepsy, Jacksonian seizure disorder, Unverricht-Lundborg disease, or photosensitive epilepsy. The epilepsy may include generalized seizures or partial (i.e. focal) seizures.

Epilepsy disorder, as disclosed herein, may be Dravet Syndrome, Lennox-Gastaut Syndrome, infantile spasm, or Ohtahara Syndrome. The epilepsy disorder may be Dravet Syndrome, Lennox-Gastaut Syndrome, infantile spasm, or Ohtahara Syndrome, or a pediatric epilepsy disorder. The pediatric epilepsy disorder may be benign childhood epilepsy, Benign Neonatal Familial Convulsions (BFNC), febrile seizures, Dravet Syndrome, Lennox-Gastaut Syndrome, infantile spasm, Ohtahara Syndrome, juvenile myoclonic epilepsy, juvenile absence epilepsy, childhood absence epilepsy (e.g. pyknolepsy), infantile spasms. In embodiments the epilepsy disorder is Dravet Syndrome. According to the invention, the epilepsy disorder is Dravet syndrome or Lennox-Gastaut syndrome.

The disclosed pediatric epilepsy disorder may be benign childhood epilepsy. The disclosed pediatric epilepsy disorder may be Benign Neonatal Familial Convulsions (BFNC). The disclosed pediatric epilepsy disorder may be febrile seizures. In embodiments according to the invention the pediatric epilepsy disorder is Dravet Syndrome. In embodiments according to the invention the pediatric epilepsy disorder is Lennox-Gastaut Syndrome. The disclosed pediatric epilepsy disorder may be infantile spasm. The disclosed pediatric epilepsy disorder may be Ohtahara Syndrome. The disclosed pediatric epilepsy disorder may be juvenile myoclonic epilepsy. The disclosed pediatric epilepsy disorder may be juvenile absence epilepsy. The disclosed pediatric epilepsy disorder may be childhood absence epilepsy (e.g. pyknolepsy). The disclosed pediatric epilepsy disorder may be infantile spasms.

In some embodiments, the epilepsy disorder may be a result of a neurological disease or injury such as, for example, encephalitis, cerebritis, abscess, stroke, tumor, trauma, genetic, tuberous sclerosis, cerebral dysgenesis, or hypoxic-ischemic encephalopathy. The epilepsy disorder may be associated with a neurodegenerative disease such as, for example, Alzheimer's disease or Parkinson's Disease. The epilepsy disorder may be associated with autism. The epilepsy disorder may be associated with a single gene mutation. The epilepsy disease may be associated with compulsive behaviors or electrographic seizures.

The epilepsy disorder may be an epilepsy disorder which is non-responsive to treatment with an antiepileptic drug (AED). The AED may be acetazolamide. The AED may be benzodiazepine. The AED may be cannabadiols. The AED may be carbamazepine. The AED may be clobazam. The AED may be clonazepam. The AED may be eslicarbazepine acetate. The AED may be ethosuximide. The AED may be ethotoin. The AED may be felbamate. The AED may be fenfluramine. The AED may be fosphenytoin. The AED may be gabapentin. The AED may be ganaxolone. The AED may be huperzine A. The AED may be lacosamide. The AED may be lamotrigine. The AED may be levetiracetam. The AED may be nitrazepam. The AED may be oxcarbazepine. The AED may be perampanel. The AED may be piracetam. The AED may be phenobarbital. The AED may be phenytoin. The AED may be potassium bromide. The AED may be pregabalin. The AED may be primidone. The AED may be retigabine. The AED may be rufinamide. The AED may be valproic acid. The AED may be sodium valproate. The AED may be stiripentol. The AED may be tiagabine. The AED may be topiramate. The AED may be vigabatrin. The AED may be zonisamide.

In some embodiments, the subject has a neurodevelopmental disorder. Representative neurodevelopmental disorders include autism spectrum disorder, Rett syndrome, fragile X, attention deficit disorder, and attention-deficit/hyperactivity disorder. In some embodiments, the neurodevelopmental disorder is a disorder known to be comorbid with seizures. As used herein, a condition “responsive to a ketogenic diet” includes, but is not limited to, epilepsy, autism spectrum disorder, Rett syndrome, fragile X, attention deficit disorder, attention-deficit/hyperactivity disorder, seizures, autism spectrum disorder, Alzheimer's disease, Huntington's disease, Parkinson's disease, amyotrophic lateral sclerosis (ALS), cancer, stroke, a metabolic disease (e.g., diabetes or obesity), a mitochondrial disorder, depression, migraines (e.g., chronic migraines), or traumatic brain injury (TBI).

In some embodiments, the subject is refractory to anti-conversant drug or anti-seizure drug. An “anti-seizure drug”, “anti-epilepsy drug”, “AED” or “anticonvulsant” are used interchangeably herein and according to their common and ordinary meaning and include compositions for reducing or eliminating seizures. Anticonvulsants include, but are not limited to acetazolamide, benzodiazepine, cannabidiols, carbamazepine, clobazam, clonazepam, eslicarbazepine acetate, ethosuximide, ethotoin, felbamate, fenfluramine, fosphenytoin, gabapentin, ganaxolone, huperzine A, lacosamide, lamotrigine, levetiracetam, nitrazepam, oxcarbazepine, perampanel, piracetam, phenobarbital, phenytoin, potassium bromide, pregabalin, primidone, retigabine, rufinamide, valproic acid, sodium valproate, stiripentol, tiagabine, topiramate, vigabatrin, or zonisamide.

In some embodiments, the compositions described herein may be administered conjointly. For example, a composition comprising a compound of Table 1 may be conjointly administered with a bacteria listed in Table 4. In some embodiments, a composition comprising a compound of Table 1 may be conjointly administered with an anticonvulsant.

TABLE 1
Compounds

3-hydroxybutyrate (BHBA)
Glucose
butyrate/isobutyrate (4:0)
valerate (5:0)
caproate (6:0)
caprate (10:0)
palmitoleate (16:1n7)
oleate/vaccenate (18:1)
10-nonadecenoate (19:1n9)
eicosenoate (20:1)
erucate (22:1n9)
tetradecadienoate (14:2)
hexadecatrienoate (16:3n3)
eicosapentaenoate (EPA; 20:5n3)
docosapentaenoate (n3 DPA; 22:5n3)
docosahexaenoate (DHA; 22:6n3)
hexadecadienoate (16:2n6)
linolenate [alpha or gamma; (18:3n3 or 6)]
dihomo-linoleate (20:2n6)
dihomo-linolenate (20:3n3 or n6)
arachidonate (20:4n6)
docosadienoate (22:2n6)
3-hydroxybutyrate (BHBA)
tryptophan
N-acetyltryptophan
kynurenine
kynurenate
anthranilate
xanthurenate
picolinate
serotonin
5-hydroxyindoleacetate
indolelactate
indoleacetate
indole-3-carboxylate
3-indoxyl sulfate
cholesterol
cholate
taurocholate
taurochenodeoxycholate
taurolithocholate 3-sulfate
ursodeoxycholate
tauroursodeoxycholate
taurocholenate sulfate
7-ketodeoxycholate

TABLE 2
Metagenomic (Gene) Pathways

AEROBACTINSYN-PWY: aerobactin biosynthesis
PWY-1861: formaldehyde assimilation II (RuMP Cycle)
POLYAMINSYN3-PWY: superpathway of polyamine biosynthesis II
PWY-5005: biotin biosynthesis II
PWY0-42: 2-methylcitrate cycle I
PWY-5747: 2-methylcitrate cycle II
PWY3DJ-35471: L-ascorbate biosynthesis IV
PWY-4981: L-proline biosynthesis II (from arginine)
PWY-5138: unsaturated, even numbered fatty acid &beta;-oxidation
GLYCOL-GLYOXDEG-PWY: superpathway of glycol metabolism and
degradation
PYRIDNUCSYN-PWY: NAD biosynthesis I (from aspartate)
PWY-5188: tetrapyrrole biosynthesis I (from glutamate)
PWY-7187: pyrimidine deoxyribonucleotides de novo biosynthesis II
PWY-7197: pyrimidine deoxyribonucleotide phosphorylation

TABLE 4
Microbiome Bacteria

Parabacteroides distasonis
Peptostreptococcaceae
Turicibacter
Bacteroides fragilis
Clostridium
Clostridium paraputrificum
Clostridium subterminale
SMB53
Clostridium colinum
Bacteroides
Clostridium neonatale
Lachnospiraceae
Dorea
Ruminococcus gnavus
Oscillospira
Clostridium ramosum
Clostridiumparaputrificum
Ruminococcus
Bilophila
Coprobacillus cateniformis
Lactococcus
Akkermansia muciniphila

In other embodiments, the subject has a condition responsive to a ketogenic diet. The condition may be Alzheimer's disease, Huntington's disease, Parkinson's disease, amyotrophic lateral sclerosis (ALS), cancer, stroke, a metabolic disease, a mitochondrial disorder, depression, migraines (e.g., chronic migraines), or traumatic brain injury (TBI). In some embodiments, the methods and compositions comprise administering to the subject a composition provided herein. In some embodiments, the condition is epilepsy, seizures, autism spectrum disorder, Alzheimer's disease, Huntington's disease, Parkinson's disease, amyotrophic lateral sclerosis (ALS), cancer, stroke, a metabolic disease (e.g., obesity or diabetes), a mitochondrial disorder, depression, migraines (e.g., chronic migraines), Rett syndrome, attention deficit disorder, fragile X syndrome, or traumatic brain injury (TBI). In some embodiments, the compositions and methods provided herein are useful in treating or preventing aging or aging-associating conditions. In some embodiments, the compositions and methods provided herein can replace the ketogenic diet in the treatment or prevention of a condition described herein; in other embodiments, the compositions and methods provided herein can be combined with the ketogenic diet. More information on conditions may be found in Stafstrom et al. (2012) Front. Pharmacol. 3:59, hereby incorporated in its entirety.

The composition may be formulated for oral delivery. In some embodiments, the composition may comprise probiotics. In some embodiments, the compositions disclosed herein are food products. The composition may be in the form of a pill, tablet, or capsule. In some embodiments, the subject may be a mammal (e.g., a human). In some embodiments, the composition is self-administered. While it is preferred for a single composition to comprise all the bacteria to be administered, it will be recognized that for any of the various embodiments described herein, the combination of bacteria can similarly be administered in multiple compositions that together comprise the combination of bacteria. For example, the invention further provides kits comprising multiple compositions that together that comprise any one of the compounds listed in Table 1 and/or any one of the bacteria that enrich any one of the gene pathways listed in Table 2 and or any one of the bacteria listed in Table 4.

In some embodiments, the composition is formulated for rectal delivery (e.g., a fecal sample). In some embodiments, the subject undergoes fecal microbiota transplant, wherein the transplant comprises a composition disclosed herein. Fecal microbiota transplantation (FMT), also commonly known as ‘fecal bacteriotherapy’ represents a therapeutic protocol that allows the reconstitution of colon microbial communities. The process involves the transplantation of fecal bacteria from a healthy individual into a recipient. FMT restores colonic microflora by introducing healthy bacterial flora through infusion of a fecal sample, e.g., by enema, orogastric tube or by mouth in the form of a capsule containing freeze-dried material, obtained from a healthy donor. In some embodiments, the fecal sample is from a fecal bank.

In some embodiments, the bacterial DNA in subject's gut microbiota is sequenced. The subject's gut bacterial DNA may be sequenced prior to administration of the composition. For example, a sample comprising bacterial DNA may be obtained from the subject, and the bacterial DNA is then sequenced for any one of the bacteria listed in Table 4, therefore measuring the presence or level of any one of such bacteria (e.g., one or more, two or more, five or more, or ten or more of the bacteria of interest) in the subject's gut microbiota. The composition disclosed herein may then be administered to the subject if the level of the bacteria is low. In some embodiments, the subject is deemed to have low levels of any one of the bacteria listed in Table 4 if less than 0.0001%, less than 0.001%, less than 0.01%, less than 0.02%, less than 0.03%, less than 0.04%, less than 0.05%, less than 0.06% less than 0.07%, less than 0.08%, less than 0.09%, less than 0.1%, less than 0.2%, less than 0.3% less than 0.4%, less than 0.5%, less than 0.6%, less than 7%, less than 0.8%, less than 0.9%, less than 1%, less than 2%, less than 3%, less than 5%, less than 7%, less than 10%, less than 20%, less than 30%, less than 40%, or less than 50% of the bacteria in the sample is the bacteria of interest. Bacterial DNA to be sequenced may be obtained through any means known in the art, including, but not limited to, obtaining a fecal sample from the subject and isolating the bacterial DNA. Bacterial DNA sequencing by any known technique in the art, including, but not limited to, Maxam Gilbert sequencing, Sanger sequencing, shotgun sequencing, bridge PCR, or next generation sequencing methods, such as massively parallel signature sequencing (MPSS), polony sequencing, 454 pyrosequencing, Illumina (Solexa) sequencing, SOLID sequencing, Ion torrent semiconductor sequencing, DNA nanoball sequencing, heliscope single molecule sequencing, single molecule real time (SMRT) sequencing, or nanopore DNA sequencing.

In some embodiments, the above methods directly act to reduce the amount of pathogenic bacteria in a subject (i.e., in the gastrointestinal tract of the subject). In some embodiments, this includes any such therapy that achieves the same goal of reducing the number of pathogenic organisms, when used in combination with the compositions described herein, would lead to replacement of the pathogenic microflora involved in the diseased state with microflora associated with a non-diseased state, or less pathogenic species occupying the same ecological niche as the type causing a disease state. For example, a subject may undergo treatment with antibiotics (e.g., antimicrobial compounds) or a composition comprising antibiotics to target and decrease the prevalence of pathogenic organisms, and subsequently be treated with a composition described herein. The treatment may also comprise an antifungal or anti-viral compound.

Suitable antimicrobial compounds include capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefmenoxime, cefmetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dirithromycin, doxycyclinepicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A1, lincomycin, lomefloxacin, loracarbef, lymecycline, meropenam, metampicillin, methacycline, methicillin, mezlocillin, micronomicin, midecamycins such as midecamycin A1, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, and protected forms thereof.

Suitable anti-fungal compounds include ketoconazole, miconazole, fluconazole, clotrimazole, undecylenic acid, sertaconazole, terbinafine, butenafine, clioquinol, haloprogin, nystatin, naftifine, tolnaftate, ciclopirox, amphotericin B, or tea tree oil and analogs, derivatives, pharmaceutically acceptable salts, esters, prodrugs, and protected forms thereof.

### Compositions

In some aspects, the invention relates to a composition (e.g., a food product or a pharmaceutical composition). Provided herein are compositions (e.g., compositions comprising any one of the compounds listed in Table 1 and a pharmaceutically acceptable carrier; or compositions comprising bacteria that enrich any one of the gene pathways in Table 2, such as any bacteria listed in Table 4, and a pharmaceutically acceptable carrier). The composition may comprise at least one, at least two, at least three, at least four, at least five, at least six at least seven, at least eight, at least nine, at least ten, at least fifteen, or at least twenty of the compounds listed in Table 1. The composition may comprise at least one, at least two, at least three, at least four, at least five, at least six at least seven, at least eight, at least nine, at least ten, at least fifteen, or at least twenty bacteria that enrich any one of the gene pathways in Table 2, such as any bacteria listed in Table 4. Any combination of the bacteria listed in Table 4 may be include in the composition.

The composition may comprise a pharmaceutically acceptable carrier. The composition may comprise probiotics. The pharmaceutical compositions disclosed herein may be delivered by any suitable route of administration, including orally, bucally, sublingually, parenterally, and rectally, as by powders, ointments, drops, liquids, gels, tablets, capsules, pills, or creams. In certain embodiments, the pharmaceutical compositions are delivered generally (e.g., via oral administration). In certain other embodiments, the compositions disclosed herein are delivered rectally.

In certain embodiments, the invention provides kits comprising multiple compositions (e.g., compositions comprising any one of the compounds listed in Table 1 and a pharmaceutically acceptable carrier; or compositions comprising bacteria that enrich any one of the gene pathways in Table 2, such as any one of the bacteria listed in Table 4, and a pharmaceutically acceptable carrier). The kits disclosed herein may comprise at least one, at least two, at least three, at least four, at least five, at least six at least seven, at least eight, at least nine, at least ten, at least fifteen, or at least twenty of the compounds listed in Table 1. The kits provided herein may comprise at least one, at least two, at least three, at least four, at least five, at least six at least seven, at least eight, at least nine, at least ten, at least fifteen, or at least twenty bacteria that enrich any one of the gene pathways in Table 2, such as any bacteria listed in Table 4. Any combination of the bacteria listed in Table 4 may be include in the composition.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, of the bacteria in the composition enrich any one of the gene pathways in Table 2, such as any one of the bacteria listed in Table 4.

Compositions described herein may be used for oral administration to the gastrointestinal tract, directed at the objective of introducing the bacteria (e.g., the bacteria disclosed herein) to tissues of the gastrointestinal tract. The formulation for a composition (e.g., a probiotic composition) of the present invention may also include other probiotic agents or nutrients which promote spore germination and/or bacterial growth. An exemplary material is a bifidogenic oligosaccharide, which promotes the growth of beneficial probiotic bacteria. In some embodiments, the probiotic bacterial composition is administered with a therapeutically-effective dose of an (preferably, broad spectrum) antibiotic, or an anti-fungal agent. In some embodiments, the compositions described herein are encapsulated into an enterically-coated, time-released capsule or tablet. The enteric coating allows the capsule/tablet to remain intact (i.e., undissolved) as it passes through the gastrointestinal tract, until after a certain time and/or until it reaches a certain part of the GI tract (e.g., the small intestine). The time-released component prevents the “release” of the probiotic bacterial strain in the compositions described herein for a pre-determined time period.

The composition may be a food product, such as, but not limited to, a dairy product. The dairy product may be cultured or a non-cultured (e.g., milk) dairy product. Non-limiting examples of cultured dairy products include yogurt, cottage cheese, sour cream, kefir, buttermilk, etc. Dairy products also often contain various specialty dairy ingredients, e.g. whey, non-fat dry milk, whey protein concentrate solids, etc. The dairy product may be processed in any way known in the art to achieve desirable qualities such as flavor, thickening power, nutrition, specific microorganisms and other properties such as mold growth control. The compositions of the present invention may also include known antioxidants, buffering agents, and other agents such as coloring agents, flavorings, vitamins, or minerals.

In some embodiments, the compositions of the present invention are combined with a carrier (e.g., a pharmaceutically acceptable carrier) which is physiologically compatible with the gastrointestinal tissue of the subject(s) to which it is administered. Carriers can be comprised of solid-based, dry materials for formulation into tablet, capsule or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. In some embodiments, the carrier is micro-crystalline cellulose (MCC) added in an amount sufficient to complete the one gram dosage total weight. Carriers can be solid-based dry materials for formulations in tablet, capsule or powdered form, and can be liquid or gel-based materials for formulations in liquid or gel forms, which forms depend, in part, upon the routes of administration. Typical carriers for dry formulations include, but are not limited to: trehalose, malto-dextrin, rice flour, microcrystalline cellulose (MCC) magnesium sterate, inositol, FOS, GOS, dextrose, sucrose, and like carriers. Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., pH 7.0). Other carriers or agents for administering the compositions described herein are known in the art, e.g., in U.S. Pat. No. 6,461,607.

Preparation may include pharmaceutically acceptable carriers. The pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compositions disclosed herein (including pharmaceutically acceptable salts thereof) or a pharmaceutical composition comprising same can also be incorporated into liposomes or administered via transdermal pumps or patches. Pharmaceutical admixtures suitable for use herein include those described, for example, in Pharmaceutical Sciences (17th Ed., Mack Pub. Co., Easton, PA) and WO 96/05309.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also disclosed herein are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations described herein can also be in the form of oil-in-water emulsions. The oily phase can be a vegetable oil or a mineral oil, described above, or a mixture of these. Suitable emulsifying agents include naturally occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Formulations may include a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are may be employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity-building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760.

In some embodiments, the composition further comprises other bacteria or microorganisms known to colonize the gastrointestinal tract. For example, the composition may comprise species belonging to the Firmicutes phylum, the Proteobacteria phylum, the Tenericutes phylum, the Actinobacteria phylum, or a combination thereof. Examples of additional bacteria and microorganisms that may be included in the subject compositions include, but are not limited to, Saccharomyces, Bacteroides, Eubacterium, Clostridium, Lactobacillus, Fusobacterium, Propionibacterium, Streptococcus, Enteroccus, Lactococcus and Staphylococcus, Peptostreptococcus. In certain embodiments, the composition is substantially free of bacteria that increase the risk of seizures or otherwise detract from the effect of a ketogenic diet. Such bacteria include Bifidobacterium bacteria. Thus, in some embodiments, the composition is substantially free of Bacteroides bacteria. A composition is substantially free of a bacterial type if that type makes up less than 10% of the bacteria in a composition, preferably less than 5%, even more preferably less than 1%, most preferably less than 0.5%, or even 0% of the bacteria in the composition.

In some embodiments, the composition comprises a fecal sample comprising at least one bacteria that enrich any one of the gene pathways in Table 2, such as any one of the bacteria listed in Table 4. In some embodiments, the fecal sample is from a fecal bank. In some embodiments, the compositions may be added to a fecal sample prior to administration to the subject.

In some embodiments, provided herein are methods of treating or preventing a condition, such as seizures, by administering a composition (e.g., a fecal sample) that is enriched for at least one bacteria that enrich any one of the gene pathways in Table 2, such as any one of the bacteria listed in Table 4. The fecal sample is enriched if at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, or at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% of the bacteria in the fecal sample is bacteria that enrich any one of the gene pathways in Table 2, such as any one of the bacteria listed in Table 4. The fecal sample is enriched if 50%, at least 52%, at least 54%, at least 56%, at least 58%, at least 60%, at least 62%, at least 64%, at least 66%, at least 68%, at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, or at least 99% of the bacteria in the fecal sample is bacteria that enrich any one of the gene pathways in Table 2, such as any one of the bacteria listed in Table 4. In some embodiments, the fecal sample is from a fecal bank. In some embodiments, the fecal sample is from a donor.

The composition may further comprise a nutrient. In some embodiments, the nutrient aids in the growth of bacteria (e.g., bacteria disclosed herein). In some embodiments, the nutrient is a lipid (e.g., lineoleic acid, stearic acid, or palmitic acid). In some embodiments, the nutrient may be conjointly administered with a composition disclosed herein. As used herein, the phrase “conjoint administration” refers to any form of administration of two or more different agents (e.g., a composition disclosed herein and a nutrient disclosed herein) such that the second agent is administered while the previously administered agent is still effective in the body. For example, the compositions disclosed herein and the nutrients disclosed herein can be administered either in the same formulation or in a separate formulation, either concomitantly or sequentially.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

### EXEMPLIFICATION

The inventors acquired microbiota from 10 pediatric epilepsy patients immediately before and 1 month after clinical treatment with the ketogenic diet. Each of the 10 pairs of samples were transplanted into cohorts of ˜14 mice each. All samples from post clinical ketogenic diet conferred seizure protection in mice.

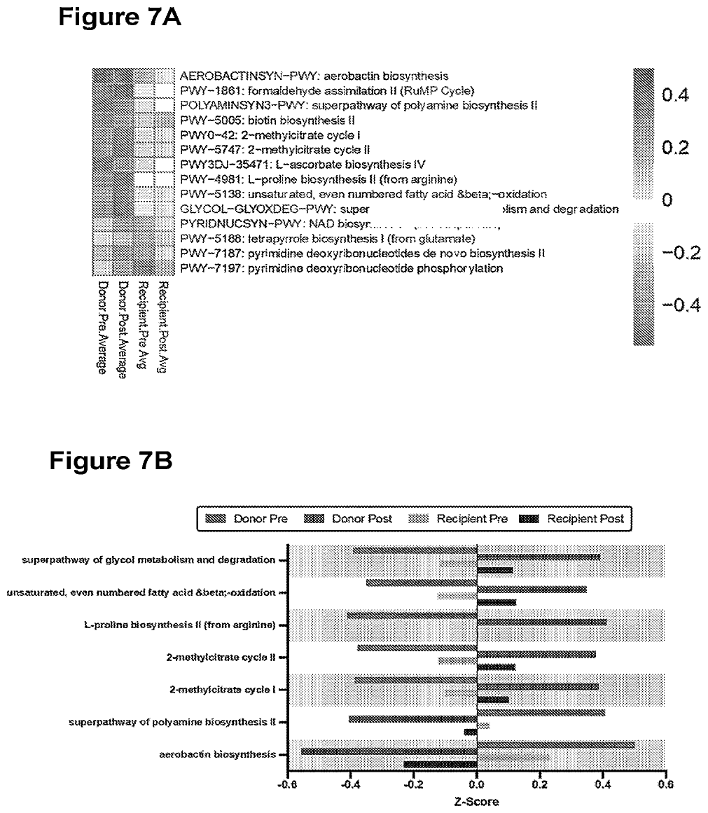

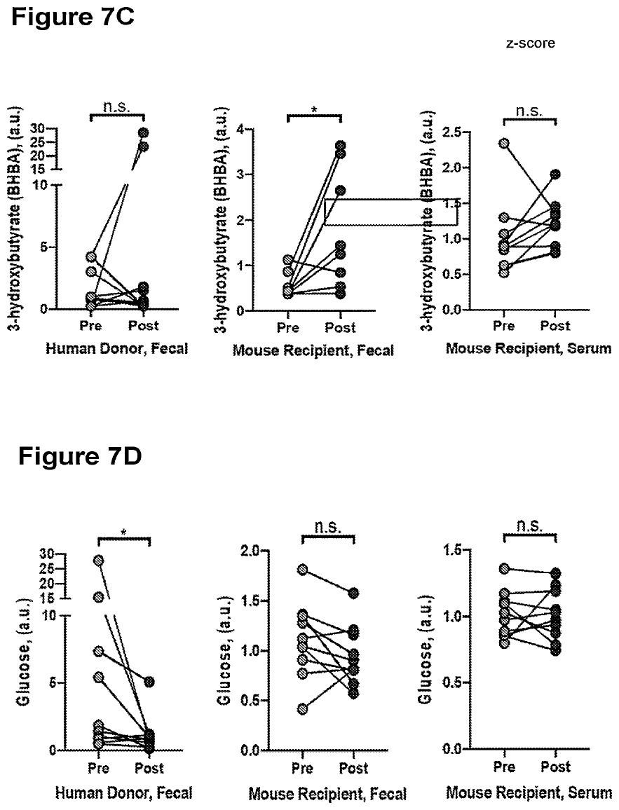

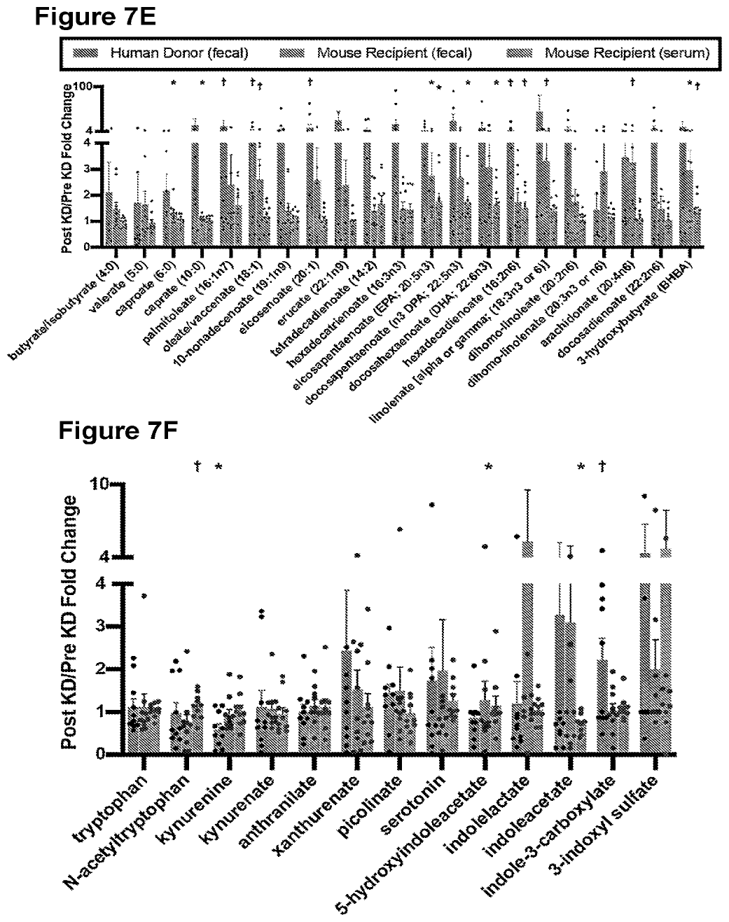

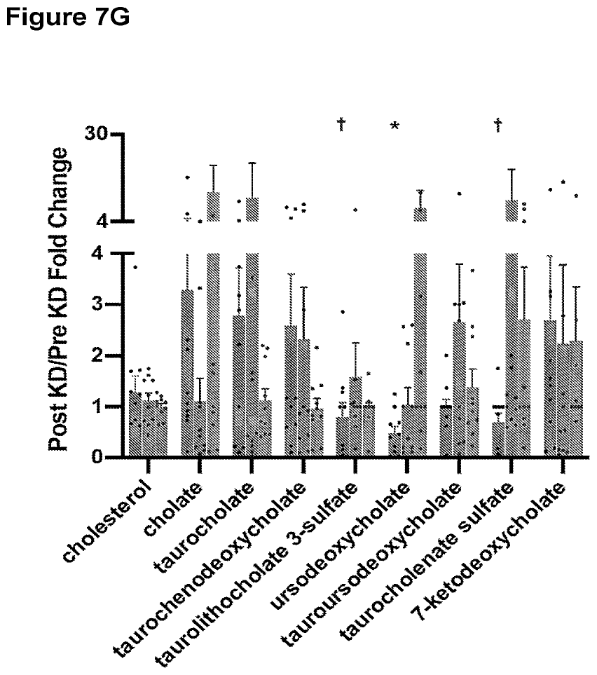

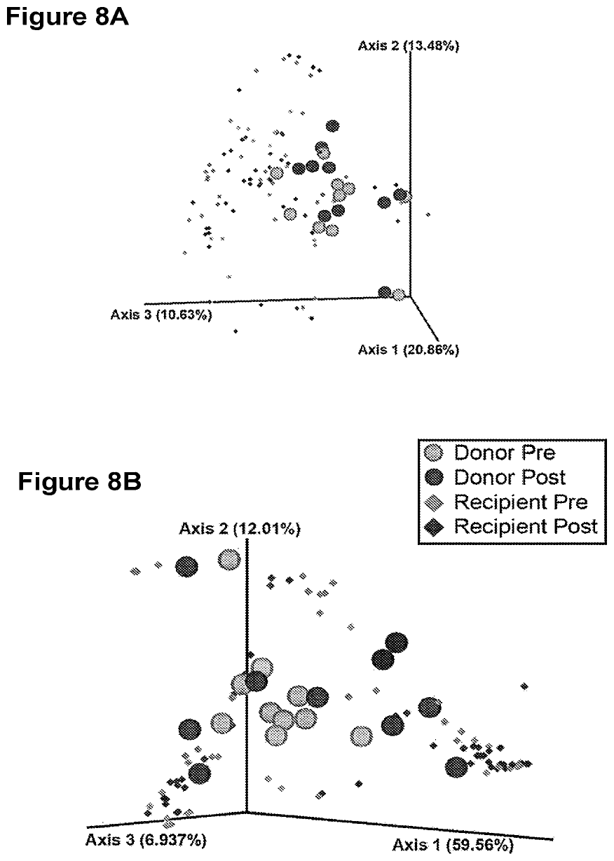

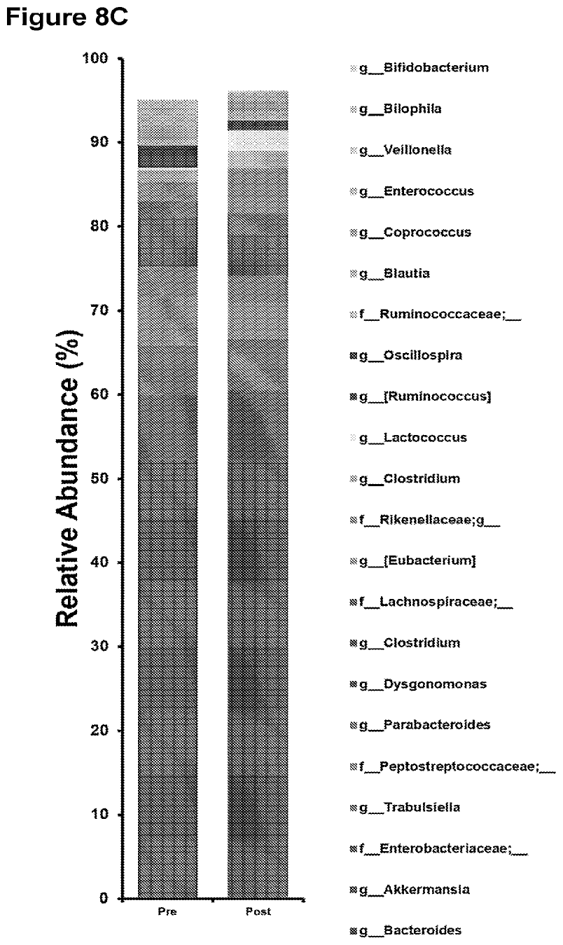

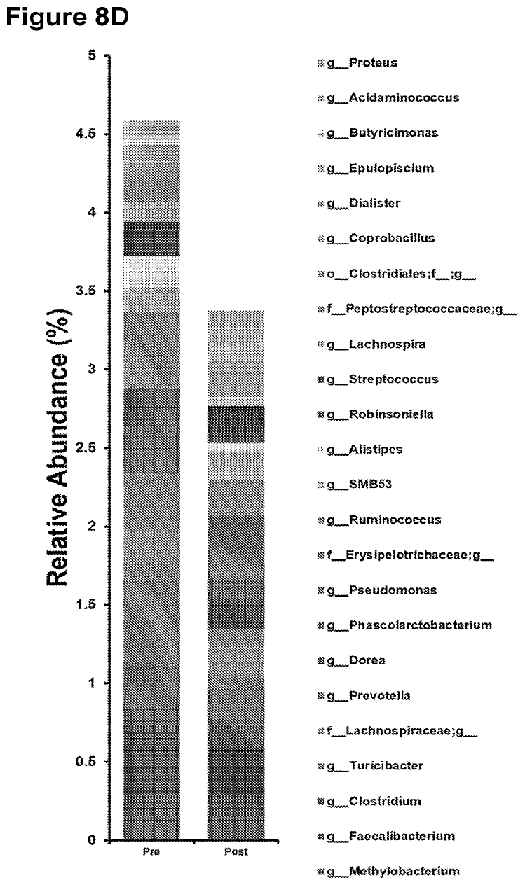

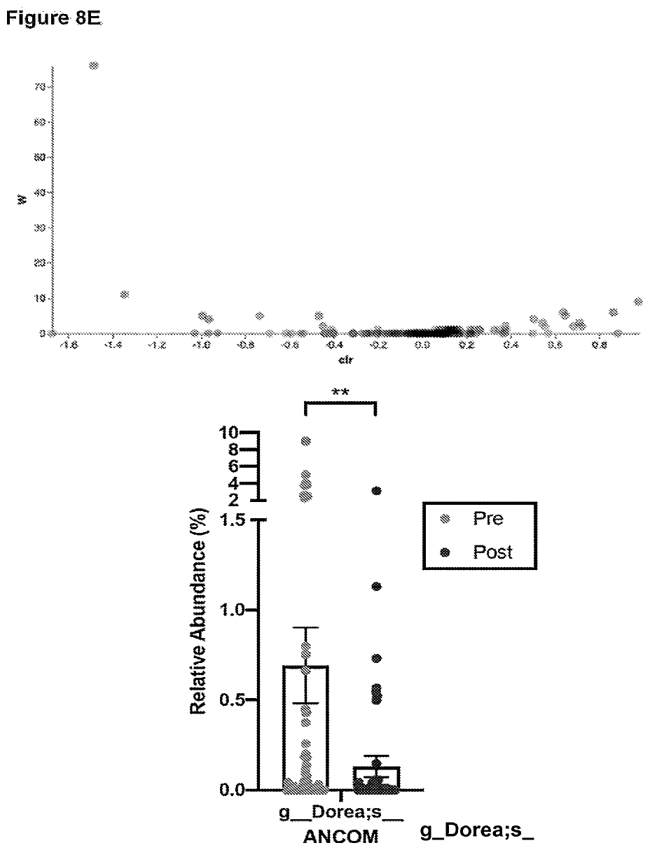

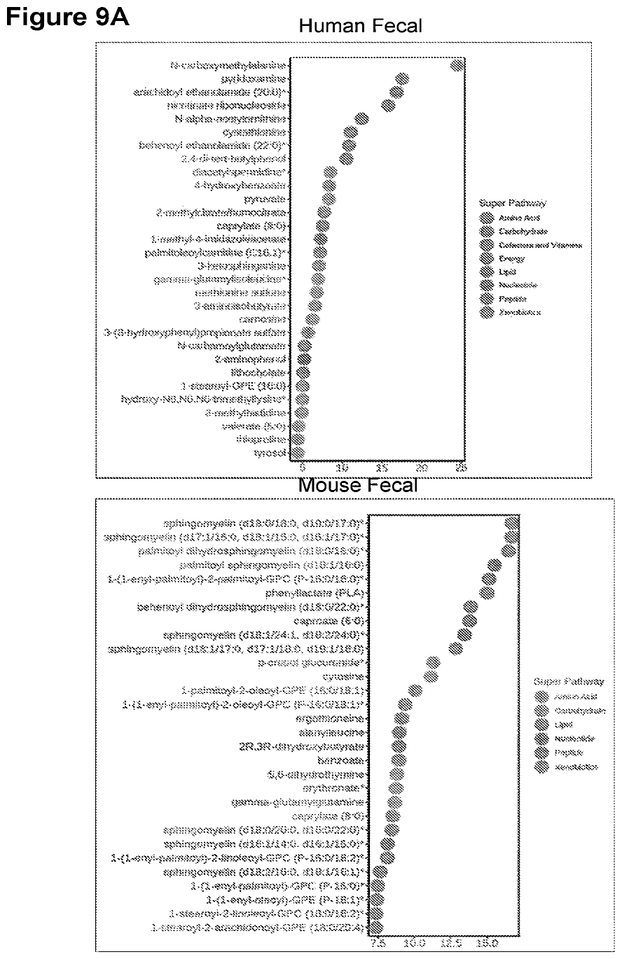

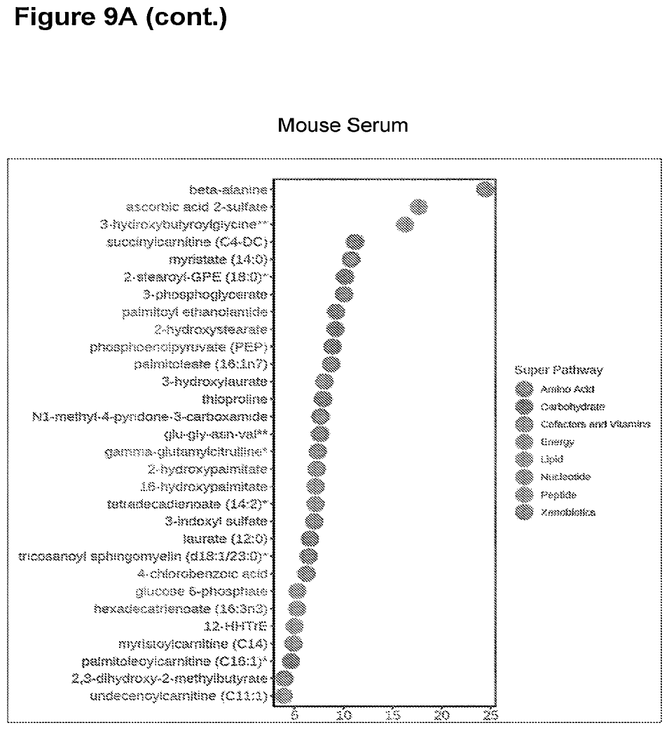

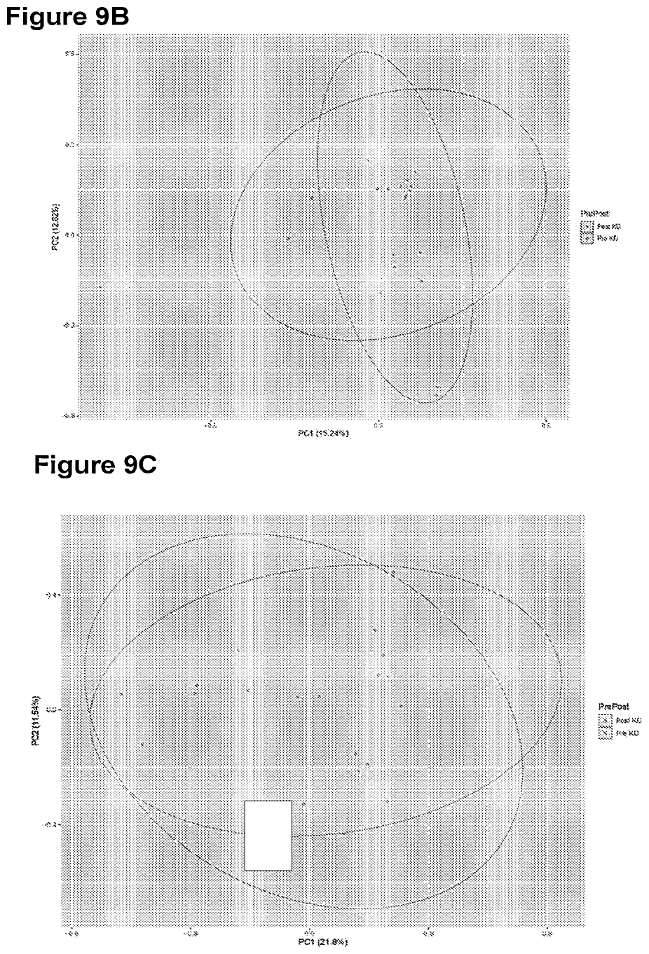

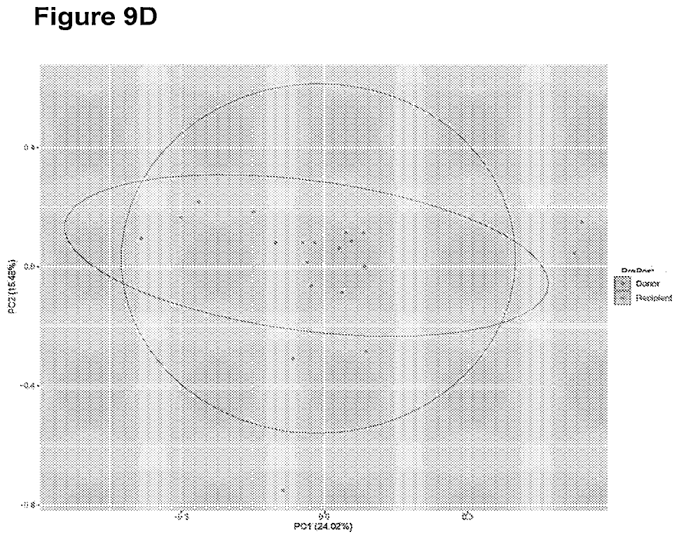

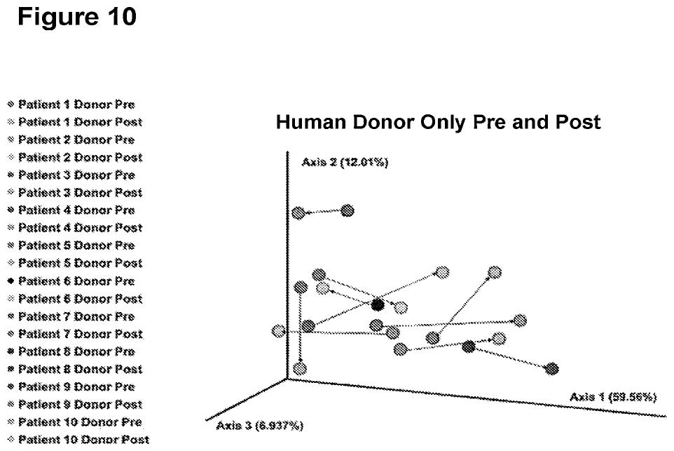

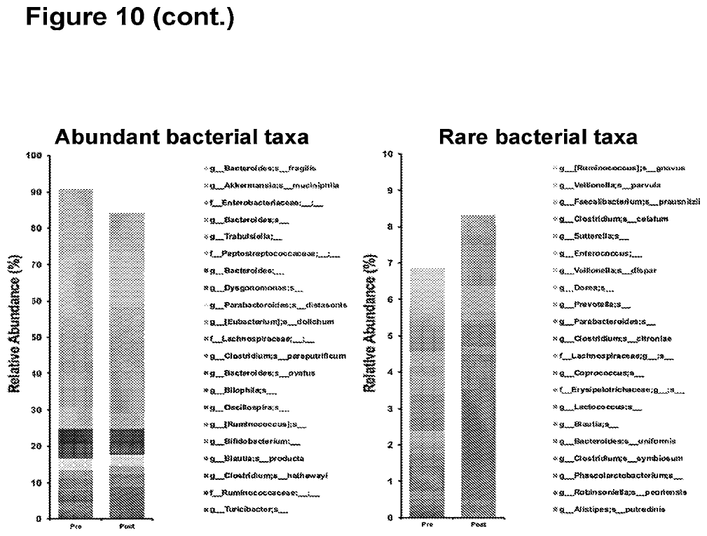

Inventors assessed bacterial functions by metagenomic sequencing and metabolomic profiling. It was found common bacterial genes and bacterial-dependent metabolites that are associated with seizure protection across all patient samples (FIG. 7). This evidence suggests that any microbial communities that enrich these gene pathways and these metabolites confer seizure protection.

TABLE 3
Pediatric Epilepsy Sample Background
Donor patient demographic data and seizure etiology (n = 10)

| Patient # | Gender | Age | KD ratio | Patient Response to KD | Epilepsy Etiology |
|---|---|---|---|---|---|
| 1 | F | 10 | 3:1 | less frequent seizures | isodicentric chromosome |
| | | | | less intense seizures | 15q duplication syndrome |
| | | | | | complicated by refractory |
| | | | | | (Intractable) epilepsy |
| 2 | F | 5 | 3:1 | — | hypoxic-ishemic |
| | | | | | encephalopathy induced |
| | | | | | seizures |
| 3 | F | 2 | 2.5:1 | less frequent seizures | trisomy 23 mosaicism |
| | | | | increased seizure intensity | |
| 4 | M | 4 | 3:1 | — | WWOX gene mutation |
| 5 | M | 3 | 1.75:1 | nonresponder | non-ketotic hyperglycinemia |
| 6 | F | 1 | 3:1 | responder, less seizures | — |
| 7 | F | 1 | 3:1 | — | — |
| 8 | M | 5 | 3:1 | — | — |
| 9 | F | 5 | 3:1 | — | — |
| 10 | F | 10 | 3:1 | — | — |

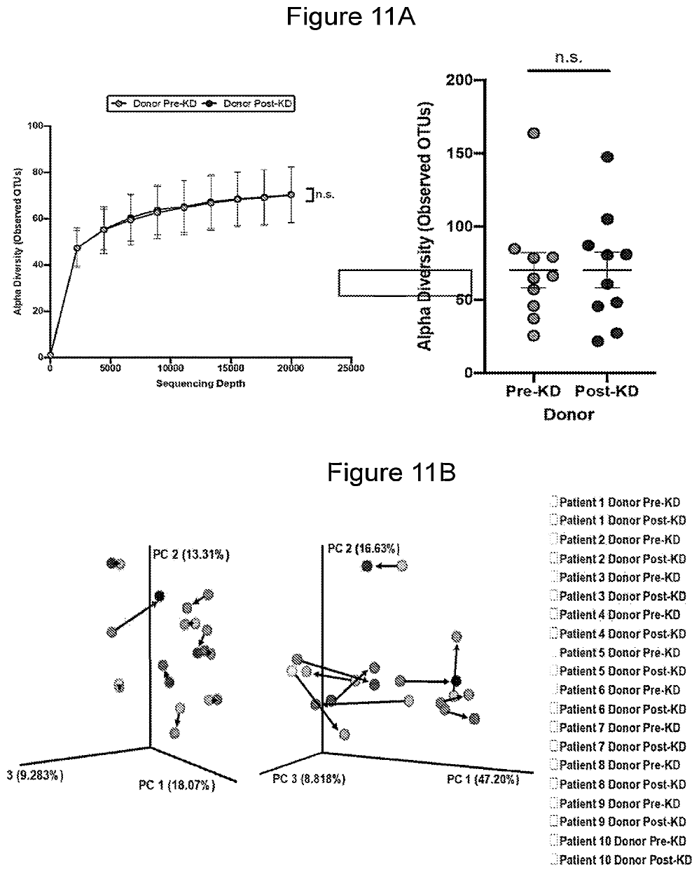

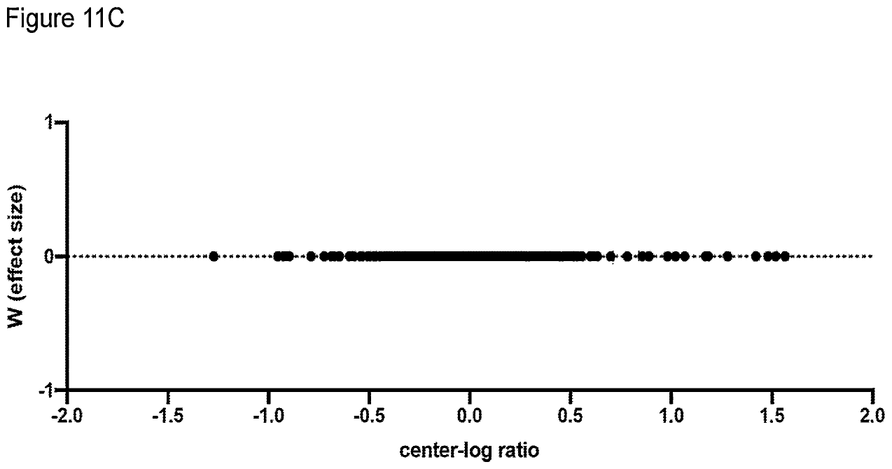

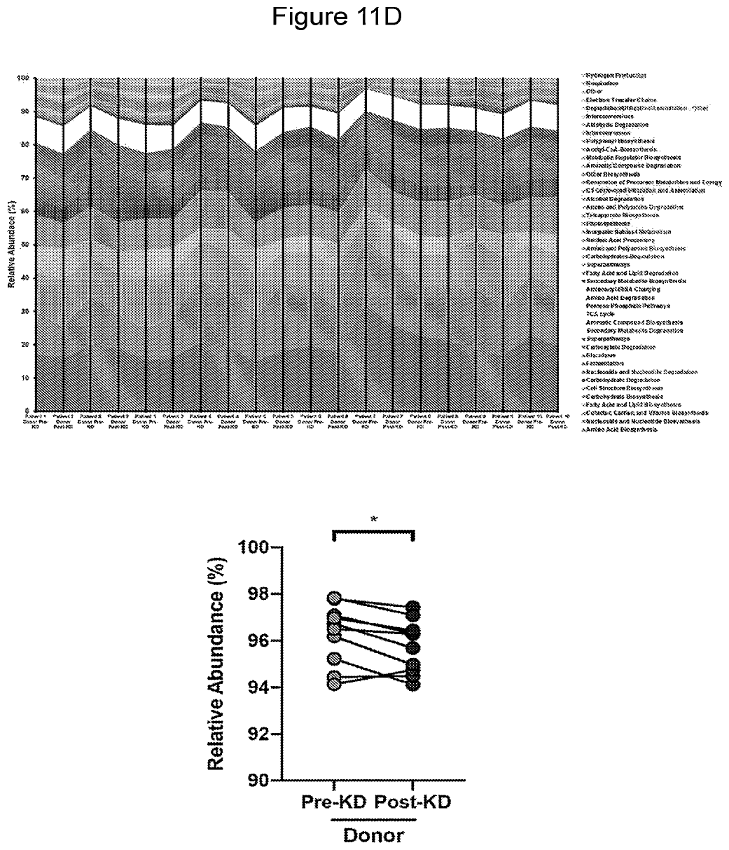

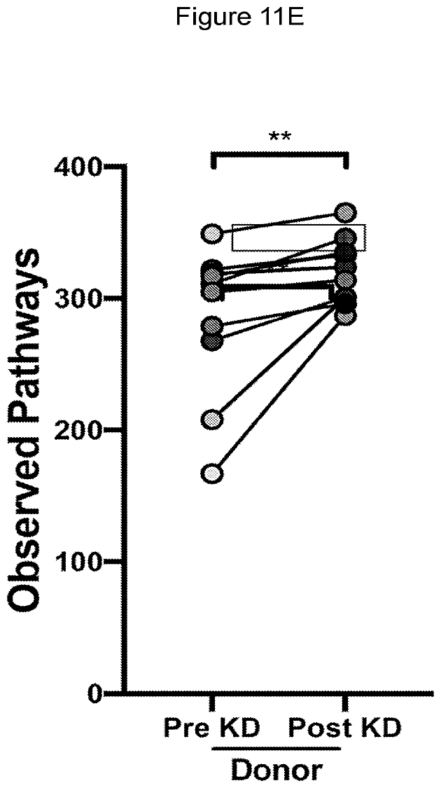

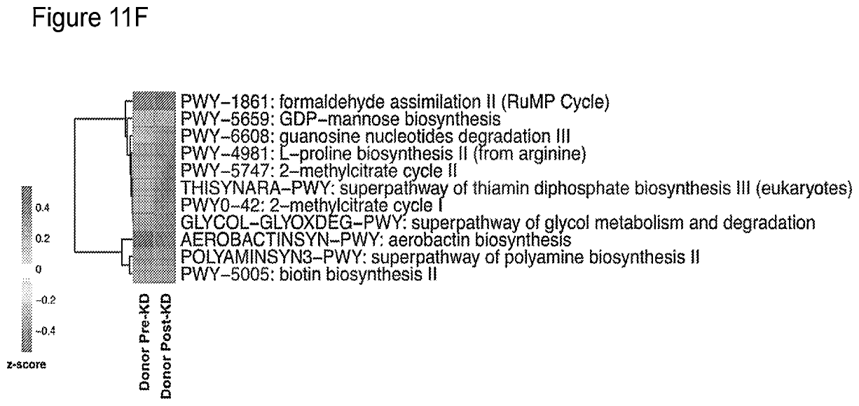

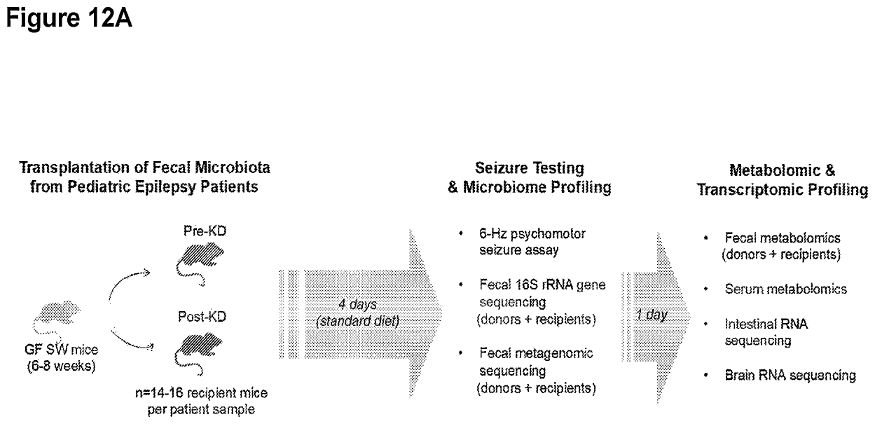

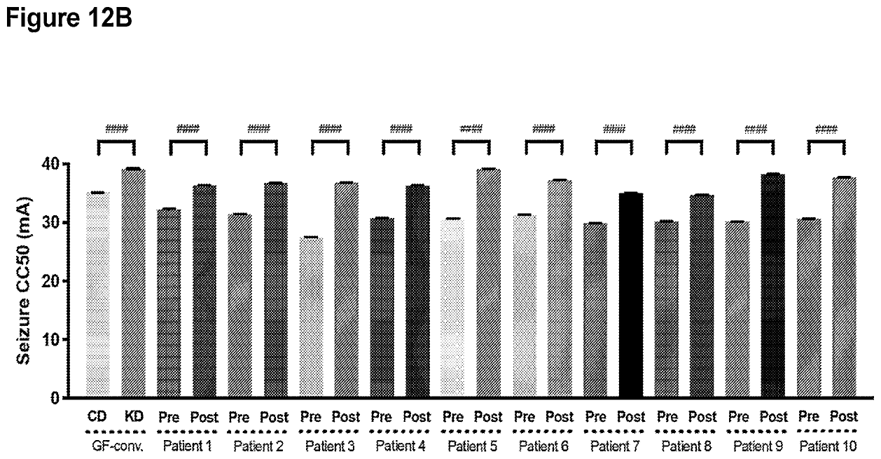

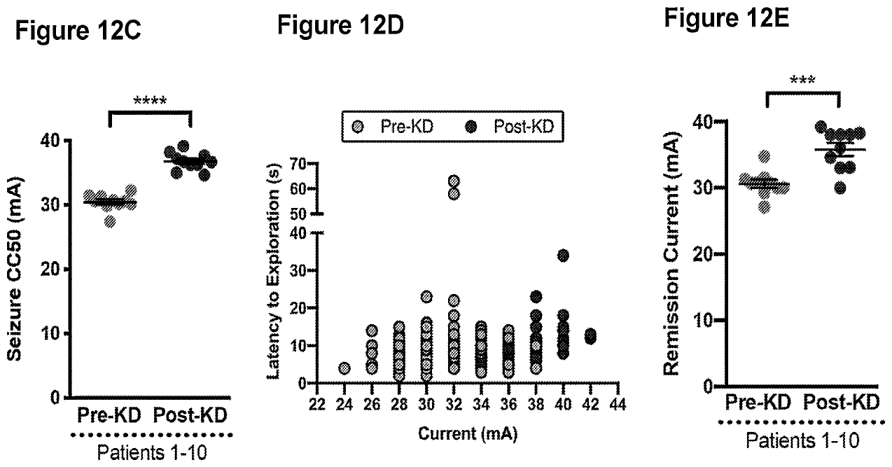

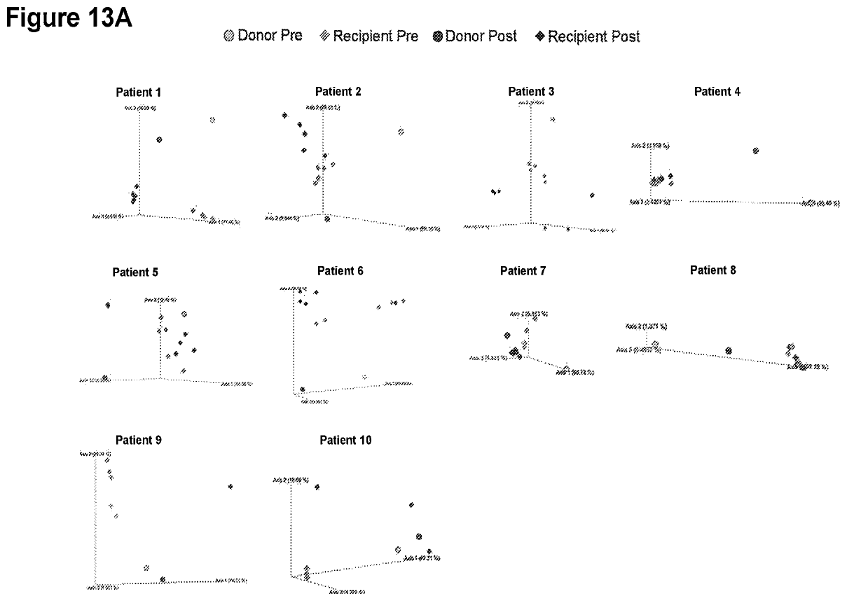

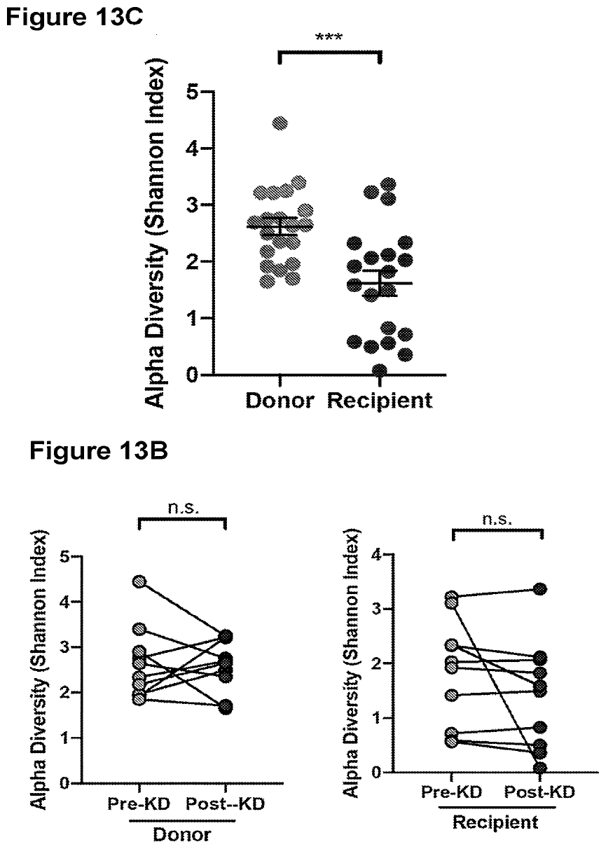

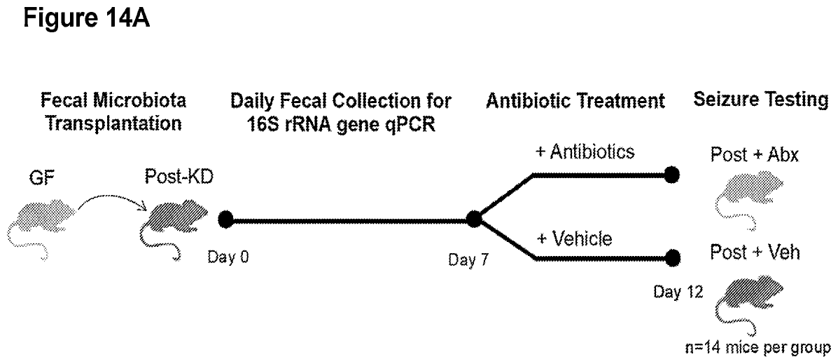

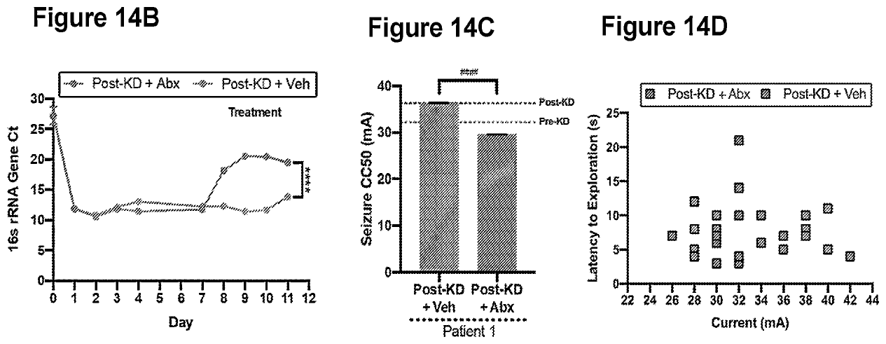

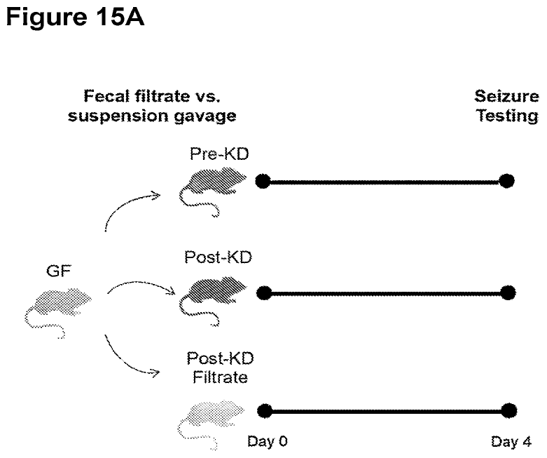

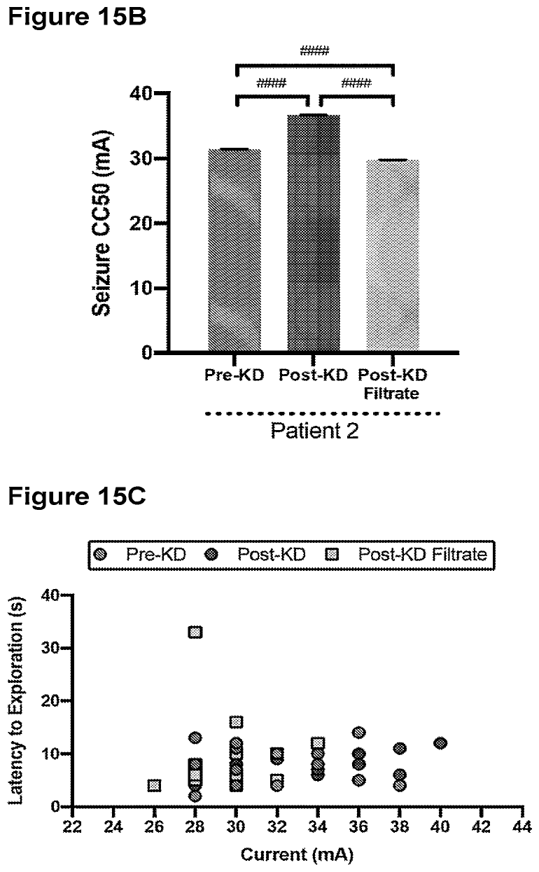

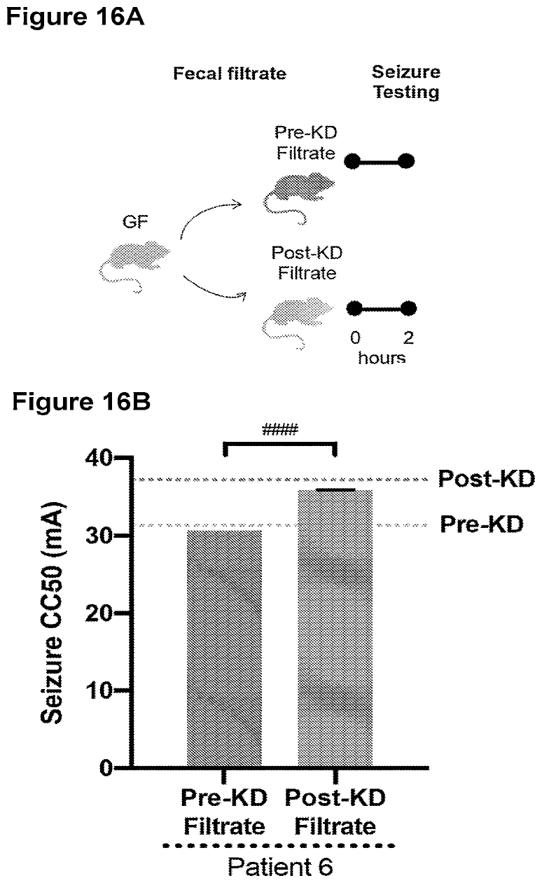

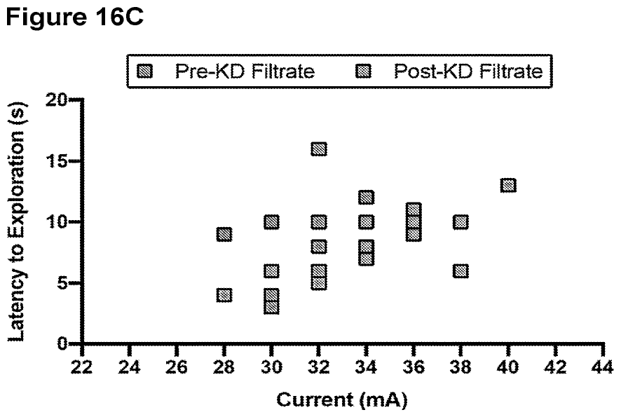

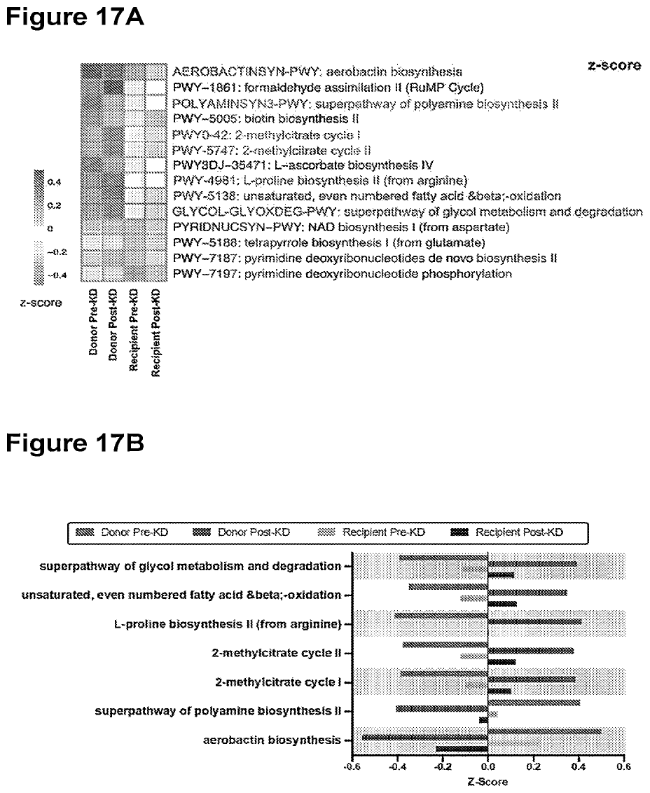

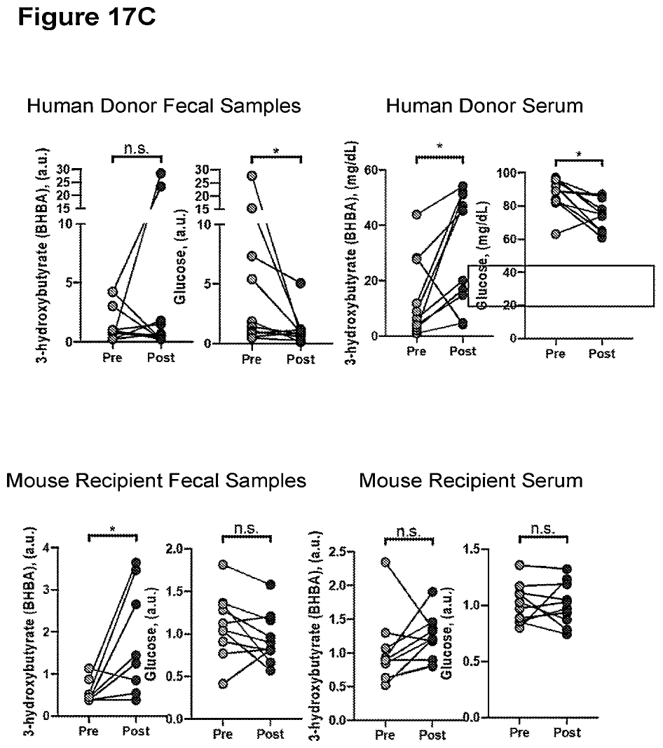

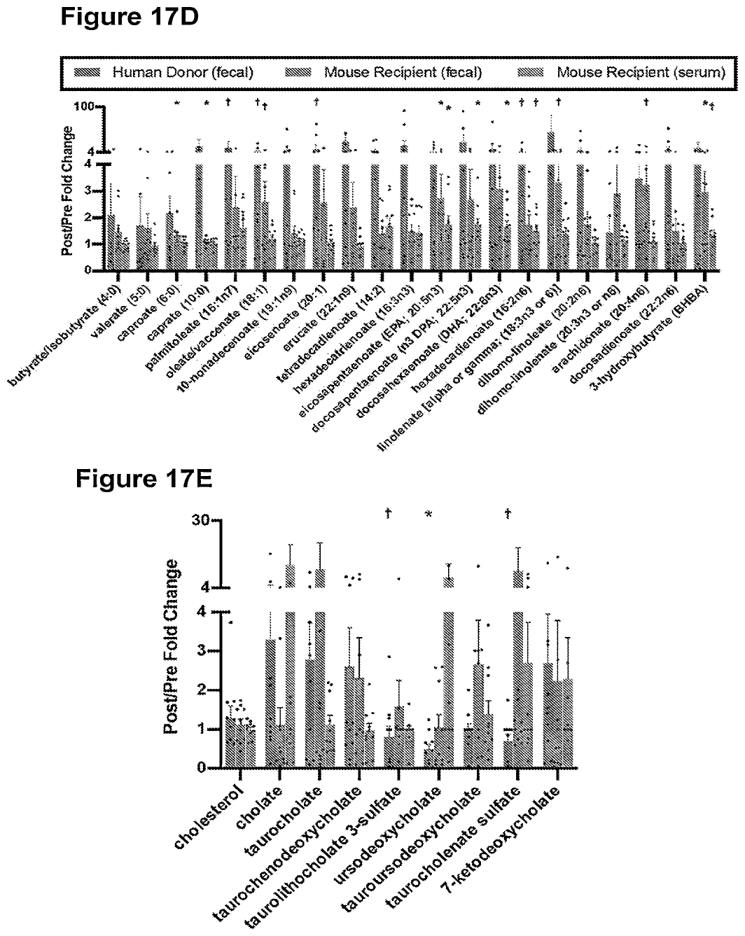

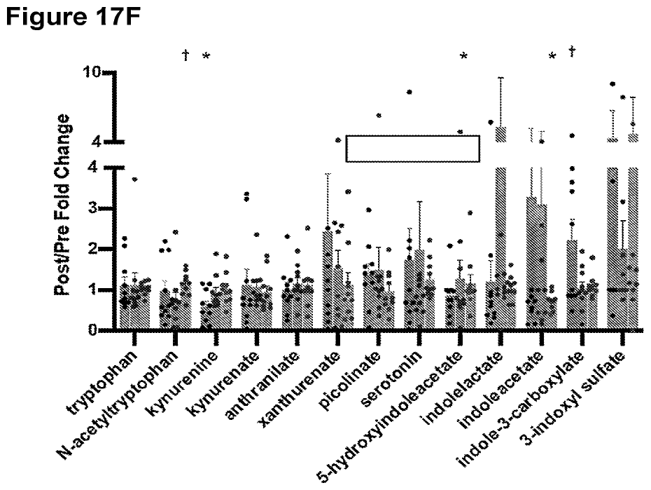

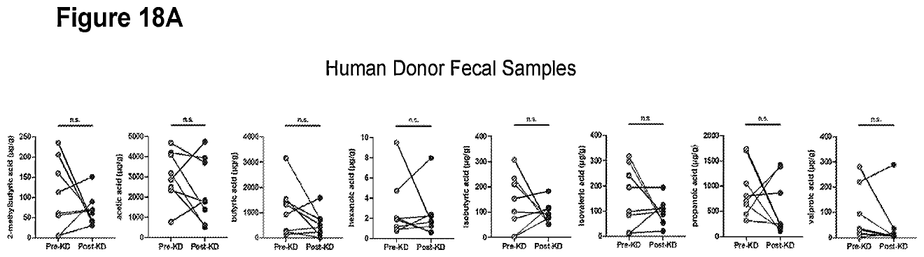

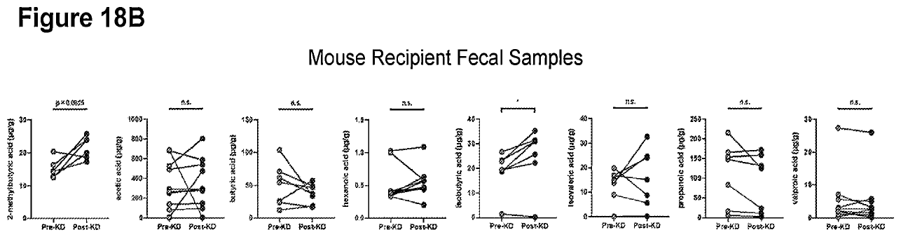

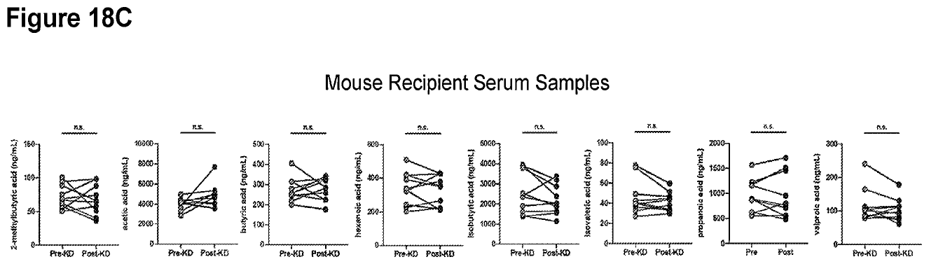

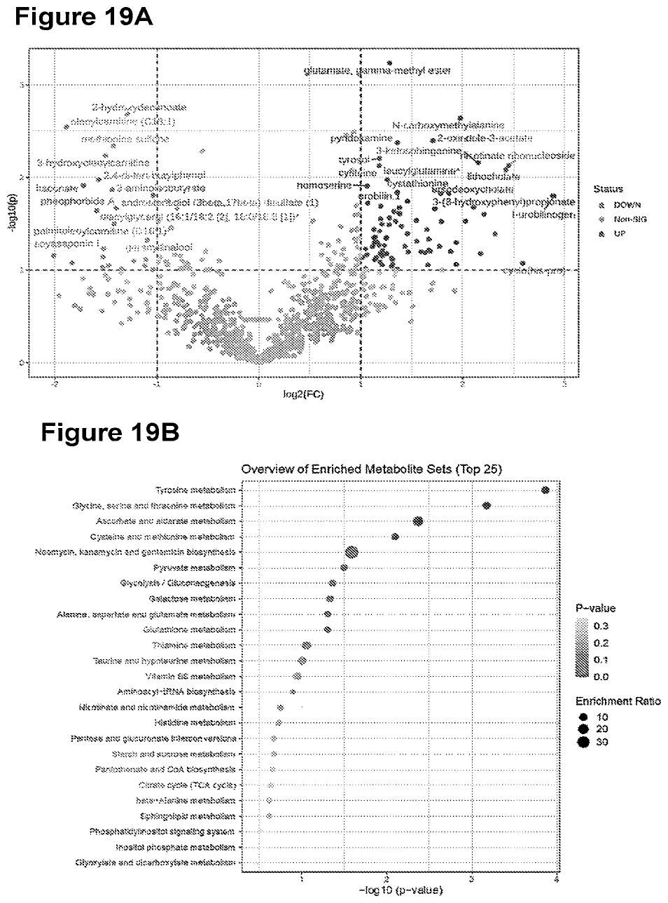

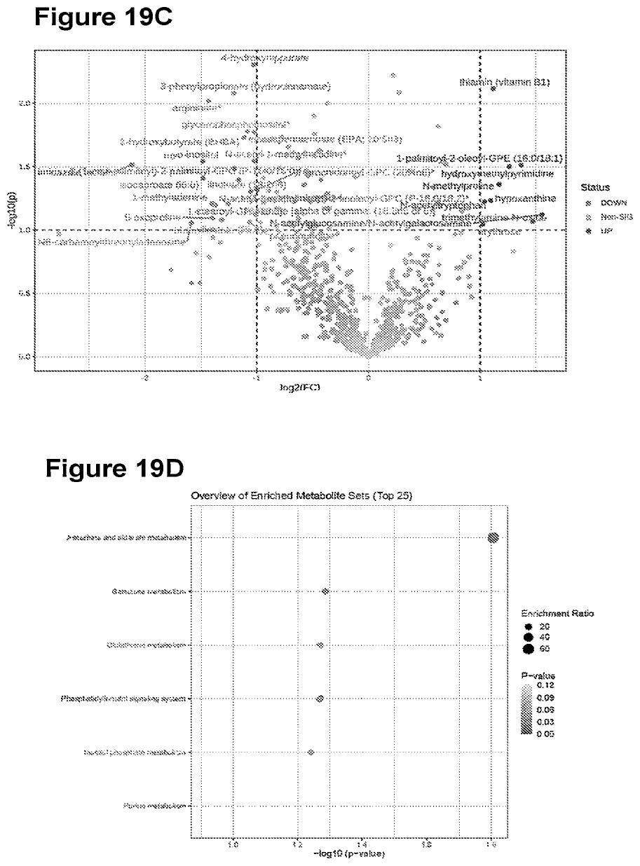

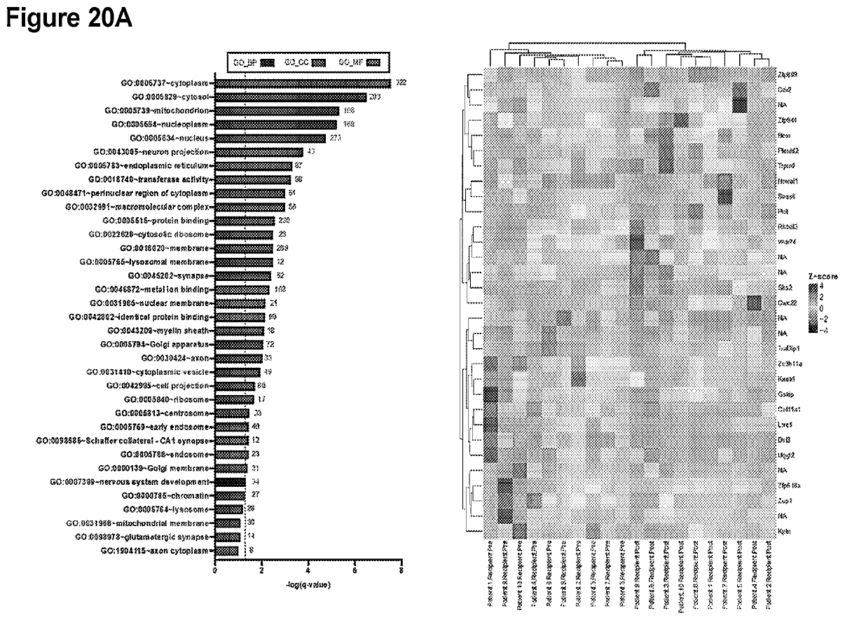

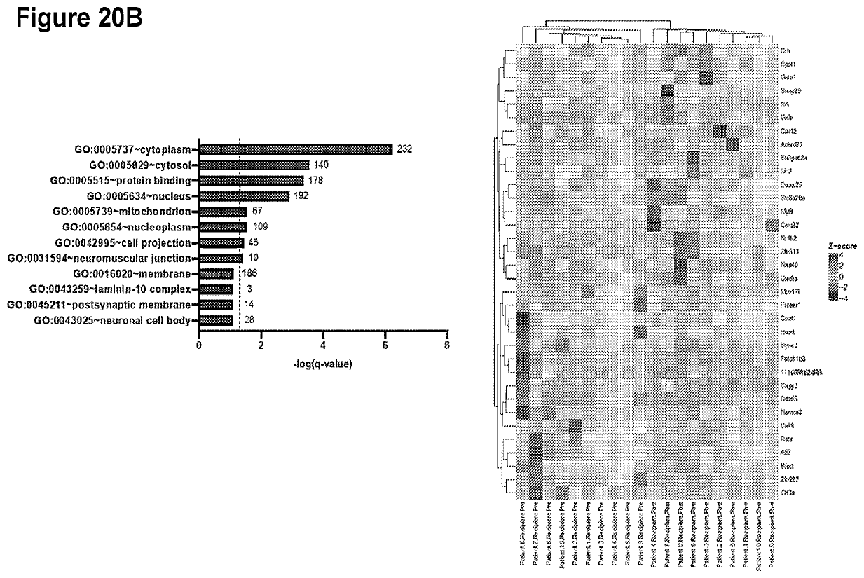

TABLE 5
Demographic and clinical information for pediatric epilepsy patients recruited for the study
shown in FIGS. 11 to 20. Patient response to KD denotes a ~50% decrease in seizures based
upon patient's parents observations and physician's notes one month after starting KD.

| Donor | | | KD | Patient | | Pre-KD | Post-KD |
|---|---|---|---|---|---|---|---|
| Patient | Sex | Age | ratio | Response to KD | Epilepsy Etiology | BHB (mg/dL) | BHB (mg/dL) |
| 1 | F | 8 | 3:1 | responder | Isodicentric | 28.3 | 4.18 |
| | | | | | chromosome 15q | | |
| | | | | | duplication syndrome | | |
| 2 | F | 4 | 3:1 | non- | Hypoxic-ishemic | 11.8 | 52.2 |
| | | | | responder | encephalopathy | | |
| 3 | F | 3 | 2:1 | responder | Trisomy 23 | 1.0 | 4.73 |
| | | | | | mosaicism | | |
| 4 | M | 1 | 2:1 | non- | WWOX gene | 2.1 | 51.2 |
| | | | | responder | mutation | | |
| 5 | M | 2 | 1.75:1 | non- | Non-ketotic | 3.1 | 16.8 |
| | | | | responder | hyperglycinemia | | |
| 6 | F | 1 | 3:1 | responder | Pyruvate | 16.3 | 20.1 |
| | | | | | dehydrogenase E1- | | |
| | | | | | alpha deficiency + | | |
| | | | | | agenesis of corpus | | |
| | | | | | callosum | | |
| 7 | F | 1 | 3.75:1 | responder | NeuroD2 mutation | 8.9 | 47 |
| 8 | M | 5 | 2.75:1 | non- | polymicrogyria | 43.9 | 54.1 |
| | | | | responder | Bilateral perisylvian | | |
| 9 | F | 5 | 3:1 | non- | Tuberous sclerosis | 27.8 | 45.3 |
| | | | | responder | | | |
| 10 | F | 10 | 2.75:1 | non- | Neuronal ceroid | 4.1 | 14.8 |
| | | | | responder | lipofuscinosis type 6 | | |
| | | | | | (CLN6/Battens | | |
| | | | | | disease) | | |

| | | | | | Medications |
|---|---|---|---|---|---|
| Donor | Pre-KD | Post-KD | Pre-KD | Post-KD | During any |
| Patient | Glucose (mg/dL) | Glucose (mg/dL) | Period AEDs | Period AEDs | other visit |
| 1 | 95 | 78 | levetiracetam | levetiracetam | CBD oil, |
| | | | | | rufinamide |
| 2 | 82 | 75 | levetiracetam | levetiracetam | — |
| 3 | 84 | 65 | ACTH, CBD oil, | — | — |
| | | | clobozam, | | |
| | | | felbamate, | | |
| | | | levetiracetam, | | |
| | | | prednisolone, | | |
| | | | topiramate, | | |
| | | | vigabratin | | |
| 4 | 83 | 61 | felbamate, | felbamate, | — |
| | | | levetiracetam, | levetiracetam, | |
| | | | rufinamide, | rufinamide, | |
| | | | zonisamide | zonisamide | |
| 5 | 97 | 75 | CBD oil, | CBD + THCa | — |
| | | | clobozam, | oil, clobozam, | |
| | | | felbamate, | felbamate, | |
| | | | levetiracetam, | levetiracetam, | |
| | | | sodium | sodium | |
| | | | benzoate | benzoate | |
| 6 | 89 | 86 | levetiracetam, | — | levetiracetam, |
| | | | vigabratin | | vigabatrin |
| 7 | 92 | 64 | felbamate | felbamate | — |
| 8 | 89 | 87 | felbamate | felbamate | — |
| 9 | 63 | 74 | CBD oil, | CBD oil, | CBD oil, |
| | | | lacosamide, | lacosamide, | lacosamide, |
| | | | levetiracetam, | levetiracetam, | levetiracetam, |
| | | | vigabratin | vigabratin | vigabratin |
| 10 | 96 | 85 | clobozam, | clobozam, | clobozam, |
| | | | levetiracetam, | levetiracetam, | levetiracetam, |
| | | | zonisamide | zonisamide | zonisamide |

We claim:

1. A method of preventing or treating a condition responsive to a ketogenic diet in a subject, comprising administering to the subject a composition comprising one or more compounds listed in Table 1.

2. A method of preventing or treating a condition responsive to a ketogenic diet in a subject, comprising administering to the subject a composition comprising bacteria that enrich any one or more of the gene pathways listed in Table 2.

3. The method of claim 2, wherein at least 30% of the bacteria in the composition are selected from the bacteria listed in Table 4.

4. The method of claim 2 or claim 3, wherein at least 50% of the bacteria in the composition are selected from the bacteria listed in Table 4.

5. The method of any one of claims 2 to 4, wherein at least 70% of the bacteria in the composition are selected from the bacteria listed in Table 4.

6. The method of any one of claims 2 to 5, wherein at least 90% of the bacteria in the composition are selected from the bacteria listed in Table 4.

7. The method of any one of the preceding claims, wherein the condition is seizures, optionally wherein subject has a neurodevelopmental condition, e.g., selected from epilepsy, autism spectrum disorder, Rett syndrome, attention deficit disorder, and fragile X syndrome.

8. The method of any one of claims 1 to 7, wherein the subject has a condition selected from Alzheimer's disease, Huntington's disease, Parkinson's disease, amyotrophic lateral sclerosis (ALS), cancer, stroke, a metabolic disease, a mitochondrial disorder, depression, migraines, and traumatic brain injury (TBI).

9. The method of any one of claims 1 to 8, wherein the subject is on a diet, and the diet is a control diet.

10. The method of any one of claims 1 to 8, wherein the subject is on a diet, and the diet is a ketogenic diet.

11. The method of any one of claims 1 to 8, wherein the subject is on a diet, and the diet is a high fat diet.

12. The method of any one of claims 1 to 8, wherein the subject is on a diet, and the diet is a low carbohydrate diet.

13. The method of any one of claims 1 to 12, wherein the composition is formulated for oral delivery.

14. The method of any one of claims 1 to 13, wherein the composition is a food product.

15. The method of claim 14, wherein the food product is a dairy product.

16. The method of claim 14, wherein the food product is yogurt.

17. The method of any one of claims 1 to 16, wherein the composition is formulated for rectal delivery.

18. The method of any one of claims 1 to 17, wherein the composition is self-administered.

19. A method of treating or preventing a condition responsive to a ketogenic diet in a subject, comprising
(a) depleting the gut microbiota of the subject,
(b) administering a composition comprising a compound listed in Table 1.

20. A method of treating or preventing a condition responsive to a ketogenic diet in a subject, comprising
(a) depleting the gut microbiota of the subject,
(b) administering a composition comprising bacteria that enrich any one of the gene pathways in Table 2.

21. The method of claims 19 and 20, wherein at least 30% of the bacteria in the composition are selected from the bacteria listed in Table 4.

22. The method of any one of claims 19 to 21, wherein at least 50% of the bacteria in the composition are selected from the bacteria listed in Table 4.

23. The method of any one of claims 19 to 22, wherein at least 70% of the bacteria in the composition are selected from the bacteria listed in Table 4.

24. The method of any one of claims 19 to 23, wherein at least 90% of the bacteria in the composition are selected from the bacteria listed in Table 4.

25. The method of any one of claims 19 to 24, wherein the composition is formulated for oral delivery.

26. The method of claim 25, wherein the composition is a food product.

27. The method of claim 26, wherein the composition is formulated for rectal delivery.

28. The method of any one of claims 19 to 27, wherein the subject is given antibiotics to deplete the subject's gut microbiota.

29. The method of any one of claims 19 to 28, wherein the subject is on a diet, and the diet is a control diet.

30. The method of any one of claims 19 to 28, wherein the subject is on a diet, and the diet is a ketogenic diet.

31. The method of any one of claims 19 to 28, wherein the subject is on a diet, and the diet is a high fat diet.

32. The method of any one of claims 19 to 28, wherein the subject is on a diet, and the diet is a low carbohydrate diet.

33. The method of any one of claims 19 to 26, wherein the composition is self-administered.

34. The method of any one of claims 19 to 33, wherein the condition is seizures, optionally wherein the subject has a neurodevelopmental condition, e.g., selected from epilepsy, autism spectrum disorder, Rett syndrome, attention deficit disorder, and fragile X syndrome.

35. The method of any one of claims 19 to 33, wherein the condition is selected from Alzheimer's disease, Huntington's disease, Parkinson's disease, amyotrophic lateral sclerosis (ALS), cancer, stroke, a metabolic disease, a mitochondrial disorder, depression, migraines, and traumatic brain injury (TBI).

36. A composition comprising any one or more of the compounds listed in Table 1 and a pharmaceutically acceptable carrier.

37. A composition comprising bacteria that enrich any one or more of the gene pathways in Table 2 and a pharmaceutically acceptable carrier.

38. The composition of claim 37, wherein at least 30% of the bacteria in the composition are selected from the bacteria listed in Table 4.

39. The composition of claim 37 or claim 38, wherein at least 50% of the bacteria in the composition are selected from the bacteria listed in Table 4.

40. The composition of any one of claims 37 to 39, wherein at least 70% of the bacteria in the composition are selected from the bacteria listed in Table 4.

41. The composition of any one of claims 37 to 40, wherein at least 90% of the bacteria in the composition are selected from the bacteria listed in Table 4.

42. The composition of any one of claims 36 to 41, wherein the composition is formulated for oral delivery.

43. The composition of claim 42, wherein the composition is a food product.

44. The composition of claim 43, wherein the food product is a dairy product.

45. The composition of claim 44, wherein the dairy product is yogurt.

46. The composition of any one of claims 36 to 41, wherein the composition is formulated for rectal delivery.
